(19) United States
(12) Patent Application Publication
(10) Pub. No.: US 2010/0028941 A1
(43) Pub. Date: Feb. 4, 2010

(54) Method for Multiparallel Construction of Host/Vector-Systems for Expression of Proteins

(76) Inventor: Gen Larsson, Lidingo (SE)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(21) Appl. No.: 12/297,879

(22) PCT Filed: Apr. 23, 2007
(86) PCT No.: PCT/SE07/50261

§ 371 Date: Mar. 17, 2009

(30) Foreign Application Priority Data
Apr. 21, 2006 (SE) 0600886-6

(51) Int. Cl. C12P 21/00; C12N 15/70; C12N 1/21

(52) U.S. Cl. 435/69.1; 435/471; 435/252.33

(57) ABSTRACT

The present invention relates to a multiparallel method for construction of host/vector-systems, comprising transforming a plurality of host strains, wherein substantially all host strains have deleterious mutations in at least one uptake system for a critical substrate needed for the growth of said host strain, with a plurality of vectors encoding at least one protein, and expressing said recombinant protein in said host strains. It further relates to host/vector systems obtainable by the method, to kits comprising the host strains and optionally the vectors, and to use of produced host/vector systems for protein expression. The method is exemplified using an E. coli strain with deleterious mutation in both ptsG and ptsM.

### FIELD OF THE INVENTION

The present invention relates to the field of cell engineering, and especially to methods and products aiming to quickly find a suitable cellular system for production of a protein or metabolite.

### BACKGROUND OF THE INVENTION

Protein Production

The HUGO project was finalised in 2001 and 30-40,000 genes in the human genome were identified. The extremely extensive work of linking this information to the location and function of the corresponding proteins is now in progress. This challenging task includes the determination of the structure of the proteins and their function in cell metabolism, how they interact and how this interaction changes over time. This new understanding will revolutionise the foundations for the design of new and even personalised drugs.

A major bottleneck in this development is the rapid and facile (cheap) production of very large numbers of proteins of unknown structure and function, in an active and functional form and in sufficient quantities for further studies.

This contains a completely new task to the protein producing community. A significant part of the challenge lies in the expected and still unknown width of inherent properties of individual members of the proteome calling for an equally wide portfolio of production route alternatives. Only a very specific and small part of these categories of proteins can be expected to be produced by already developed production systems. Further, since the production method cannot be known on basis of the gene structure, this calls for a randomised procedure in order to rapidly find the best method of production and the randomised procedure, in turn, calls for a multiparallel production technique for each product. Thus, both the industrial and the academic sectors would benefit from such a procedure. Industry would use it for the early development steps of drug discovery i.e. for protein target production, but also to rapidly produce proteins for determination of structure and function. Further, when a biopharma company develops a production method for their protein product, there is a need to rapidly screen a number of constructs in order to find the best expression system for further process development and scale-up. This cannot today be done under “process-like” conditions.

Research on high throughput protein production is fragmented in Europe. Intensive activities have been initiated in many laboratories to make use of the advances in genomics. Researchers in need of specific proteins develop ‘home made’ expression systems, not taking full account of the expertise available among researchers actually specialising in protein production or the research findings from this field of science and it is questionable how far such a development can reach.

There thus exist a need to find an overall solution to this problem and this is one aim of the present invention.

Cultivation

Cultivation is a biocatalytic process that is used all over the world to produce biological substances, for example recombinant proteins. Two of the main modes of cultivation operation are batch and fed-batch. In batch cultivation all substrate components are added from the beginning and results in high concentrations at all times. This makes the reaction rate unrestricted with respect to substrate concentration. The growth rate and the specific substrate consumption are at their maximum values during most of the cultivation. In fed-batch cultivation one substrate component is added in such a way that its concentration is reaction rate limiting. This means that the reaction rates can be controlled via the feed rate of the limiting component. Specifically, the growth rate, the oxygen consumption rate, the overflow of metabolic byproducts and the specific substrate consumption are limited during most of the cultivation. The fed-batch cultivation is the most commonly used production technique and batch cultivation is rarely used, and there are two main reasons for that. The first is that the substrate limitation in fed-batch offers a tool for reaction rate control, which means you can avoid engineering limitations due to, for example, the bioreactor limitations in oxygen transfer. The second reason is that you have a metabolic control by which catabolite repression and sugar over-flow metabolism can be avoided. The fed-batch technique is the preferred and only available tool to create high cell densities and thus higher total protein production. On the other hand, the fed-batch technique is technically more complicated, more expensive and requires more supervision than the batch technique. This leads to that fedbatch cultivation needs a technically skilled operator and to a limitation in the size of the reaction vessel due to the control technology and the costs of this. This powerful technique can thus not today be used in the multiparallel production format which is required.

One aim of the present invention is thus to provide a technology for protein expression by cultivation having the advantages of fed-batch cultivation, but the simplicity of batch cultivation.

### SUMMARY OF THE INVENTION

The research of the present inventor has now led to the creation of a technology platform which should allow the unknown protein from any gene to be produced in the required multiparallel randomised format.

The present invention achieves the above identified aims by providing a collection of bacterial or yeast cells, or other eukaryotic cell lines, that can be used in batch culture, where all nutrients are in excess, but perform according to the characteristics of fed-batch culture where the cells experience a growth limitation. This is achieved by introducing one or more deleterious mutations in an uptake system for a substrate critical for the growth of the cell. This results in a restriction of the availability of the critical substrate to the cell and thus to a restricted growth and lower oxygen consumption, acetic acid production and heat production. This would be theoretically comparative to fed-batch processing and would thus allow the accumulation to higher cell densities. This strategy would also give another benefit. Several authors have shown that the glucose feed rate is a major parameter influencing not only the specific productivity but also the solubility and proteolysis rate of a protein product. In this way we could therefore further increase the number of production technologies and widen the techniques to be used in the high throughput format. However, a lot of the disadvantages of fed-batch culture, e.g. need for extensive monitoring and control of culture parameters, are avoided.

In a first aspect, the present invention relates to a multiparallel method for construction of host/vector-systems, comprising transforming a plurality of host strains, wherein substantially all host strains have deleterious mutations in at least one uptake system for a critical substrate needed for the growth of said host strain, with a plurality of vectors encoding at least one protein, and expressing said recombinant protein in said host strains.

The host strains may be prokaryotic, such as strains of Escherichia coli-or Bacillus spp, or eukaryotic, such as Pichia pastoris, Saccharomyces cerevisiae or Saccharomyces carlsbergensis, or Schizosaccharomyces pombe. One uptake system which may be mutated in E. coli is the phosphoenolpyruvate dependent carbohydrate phosphotransferase system (PTS), e.g. the genes ptsG and ptsM. The corresponding systems may be mutated in the other organisms.

In one embodiment of the invention, the host strain comprise a vector providing the deleteriously mutated member of an uptake system under control of an inducible promoter. The expression of the member, and thus the efficiency of the uptake system, may then be regulated by adding a substrate inducing the promoter to the growth media. This gives a further possibility to vary growth parameters in the system. A variety of inducible promoters is well known to the person skilled in the art and may be selected according to the specific system used.

In a further aspect the invention relates to a kit of parts comprising a plurality of host strains for use in the method according to the first aspect. The kit of parts may also include the plurality of vectors. The vectors may be provided either with the relevant protein encoding sequence or with a site into which it is easy to insert a protein encoding sequence, such as a restriction enzyme cleavage site.

In yet a further aspect, the invention relates to a host/vector-system obtained or obtainable by the method according to the first aspect and to the use of such a system in recombinant expression of proteins.

Definitions

All words and terms used in this application are intended to have the meaning usually given to them by the person skilled in the art. However, for the sake of clarity, a few terms are explicitly defined below.

A “host strain” is any bacterial or yeast strain or animal or plant cell line that can be transformed with an expression vector and used in this invention.

An “expression vector” is any nucleic acid molecule encoding at least one polypeptide and having the necessary regulatory sequences to allow this polypeptide to be expressed in the selected host strain.

An “uptake system” is a cellular system comprising at least one transporter protein facilitating the transport of a substrate into a cell.

A “deleterious mutation in an uptake system” is a mutation in the coding sequence or any regulatory sequence for a member of an uptake system, or any other mutation, which decrease the activity, performance or expression of that member and thus decrease the efficiency of the uptake system.

“Process-like conditions” or “production conditions” describes the conditions which are used in the final production process to reach an economically viable production process not only the expression of protein from a single cell. These are exemplified under the headline “Cultivation” but includes further all parameters used to control the microenvironment in which the cells are growing. The final productivity, or process economy, is set by the individual cell expression rate multiplied by the number of cells. Under production like conditions these two parameters are optimised in parallel.

A “randomised format” comprises a number of production elements put together in a randomised fashion for each product that is to be produced. Typically these elements contains expression strategies for example for transcription, translation, protein folding and peptide sequences for protein purification.

A notion written in the format of “ptsM” or “PtsM”, or alternatively “ptsM- or Pts-” refer to a deletion of the gene and consequently the expression of the corresponding protein, respectively.

### BRIEF DESCRIPTION OF THE DRAWINGS

FIG. 1: The principal design of one set of vectors usable in the invention. They have an origin of replication which results in a low (Low Ori) or high (High Ori) copy number, a malK or lacUV5 promoter (PmalK and PlacUV5, respectively), optionally a histidine or Zbasic purification tag (His, Zb), an optional protein to be expressed (ProtX) flanked by proteolytic (3C) cleavage sites, optionally a reporter protein (GFP or Bla), optionally a signal peptide (OmpA), an antibiotic resistance (Amp (ampicillin), Cm (Chloramphenicol) or Km (kanamycin)) and for the lacUV5 promoter a repressor (lacI). The vector used in the experimental part is shown at the bottom of the figure.

FIG. 2: Shows that the cell growth is restricted by the mutation in batch cultivation in minimal salts medium. Cell accumulation per ml measured by OD(600) during batch cultivations with and without plasmid. AF1000=wildtype, PPA668=ptsM, PPA652=ptsG, PPA689=ptsG,ptsM.

FIG. 3: Shows the exact restriction of the cell growth by the mutation in batch cultivation in minimal salts medium. Cell mass accumulation in g/l for batch cultivations with and without plasmid. Exponential curve fits have been fitted to the data points. The specific growth rate μ is seen from the curve fit according to y=k*e^{μ}^{ₓ}, AF1000=wildtype, PPA668=ptsM, PPA652=ptsG, PPA689=ptsG,ptsM.

FIG. 4: Shows the production of a selected protein in one of the mutated strains in minimal salts medium. The β-gal activity in U/ml (A) and U/mg dry weight (B) during two batch cultivations with plasmid. C; The specific production rate qₚ in U/(mg*h) during the batch cultivations with plasmid. AF1000=wildtype, PPA668=ptsM.

FIG. 5: Shows that overflow metabolism can be restricted in batch cultivation with the mutants during growth in minimal salts medium. Acetic acid accumulation (A) in (mg/l)/(g/l dry weight) and (B) in mg/l, during batch cultivations with and without plasmid. AF1000=wildtype, PPA668=ptsM, PPA652=ptsG, PPA689=ptsG,ptsM.

FIG. 6: Shows the growth restrictions of the mutants in complex medium. A; OD(600) for shake flask cultivations with ptsG and the double mutant, ptsG,ptsM, with plasmid in yeast and minimal medium. Cells were first grown in yeast medium, and at time t=8.25 directly after sample collection 18 ml of cells were withdrawn, centrifuged and immediately used as inoculum in new minimal medium shake flasks. The OD(600) was measured again, both in yeast and minimal medium flasks from time t=25 h. B; The β-gal activity in U/mg for the shake flask cultivations is plotted in FIG. 5B. PPA652=ptsG, PPA689=ptsG,ptsM.

FIG. 7: Shows the accumulation of a selected product under growth in complex medium A; OD(600) during shake flask cultivations with ptsG and ptsG,ptsM including plasmid. B; The β-gal concentration in U/mg for shake flask cultivations with ptsG and ptsG&,ptsM including plasmid. In the cAMP test with ptsG, 4 mM of cAMP was added to the medium directly after the first sample collection at t=18.58. PPA652=ptsG, PPA689=ptsG,ptsM. glu=glucose/maltose shake flask, aa=amino acid shake flask, ctl=control shake flask in minimal salts medium.

FIG. 8: Biomass accumulation as a function of cultivation time. 8a) Comparison of the wild type cell (WT) and the mutants grown in batch cultivation. Circles: WT strain, squares: PTS_{Man} strain, diamonds: PTS_{Glc} strain, triangles: PTS_{GlcMan} strain. 8b) The WT strain cultivated in fed-batch mode at the feed rates corresponding to the mutated strains which showed a reduced growth rate compared to the WT. Time=0 corresponds to the feed start. Diamonds: WT corresponding to PTS_{Glc} strain and triangles: WT corresponding to PTS_{GlcMan} strain.

FIG. 9: Accumulation of acetic acid as a function of cultivation time. Comparison of the wild type cell (WT) and the mutants grown in batch cultivation. Circles: WT, squares: PTS_{Man}, diamonds: PTS_{Glc}, triangles: PTS_{GlcMan}.

FIG. 10: Specific oxygen consumption rate at different growth rates in batch and fed-batch cultivation. The comparison is similar to the one in FIG. 9. In the figure is indicated the batch cultivations (WT, PTS_{Glc}, PTS_{Man} and PTS_{GlcMan}). The fed-batch cultivations of the WT are indicated by “WT corresponding to PTS_{Glc}” and “WT corresponding to PTS_{GlcMan}”, and for these cultivations time=0 corresponds to the feed start.

FIG. 11: Specific production rates of β-galactosidase during batch cultivation of the WT strain and the mutants as a function of the cultivation time. Production is constitutively expressed by use of a lacI_{q} mutation. Circles: WT, squares: PTS_{Man}, diamonds: PTS_{Glc}.

FIG. 12: Comparison of production rate of the PTS_{Glc} mutant and the WT strain grown in fed-batch at a growth rate corresponding to the mutant. The production of β-galactosidase is induced by IPTG at time zero. Circles: WT (fed-batch), diamonds: PTS_{Glc} (batch).

FIG. 13: High density cultivations in batch format of a) the WT strain and b) the PTS_{GlcMan} mutant strain. Closed symbols: specific growth rate approximated by a solid straight line, open symbols: acetic acid concentration, solid line over the whole production interval: the specific oxygen consumption rate, q_{O2}.

### DETAILED DESCRIPTION OF THE INVENTION

According to the present invention, it is possible to rapidly construct a large number of host/vector-systems by which a large number of multiparallel production systems can be achieved. The method comprises combining the strains with deleterious mutations in an uptake system with a plurality of vectors, i.e. a vector library.

The vectors may be designed by the user or may be commercially available. It is preferable that the vectors individually comprise the following sequences: an origin of replication, a promoter, a protein encoding sequence and optionally a signal peptide, a reporter protein, a purification tag and/or an antibiotic resistance gene. Care is taken so that the antibiotic resistance on the vector is compatible to the antibiotic resistance used to select for the mutated strains if this technique is used to construct the strains. These sequences are provided in different combinations in the vector library and introduced into each of the host strains to give a plurality of host/vector-combinations.

The invention will now be described in relation to a specific embodiment in which the host strains are four strains of E. coli.

Strains:

Four strains are used in the experimental part, one wild-type (wt) and three mutants derived from the wt. The wild-type is AF1000, derived from MC4100 and relA+, which means that this strains carry all components for full operation of the stringent response, but the genotype is lac− and does not contain any of the genes of the lactose operon. The three mutants are PPA652, PPA668 and PPA689. They have mutations in parts of the phosphoenolpyryvate:phosphotransferase system (PTS) with respect to the uptake of glucose and mannose and can therefore not take up these substrates at the maximum rate which result in growth at submaximum rate in a medium containing high amounts of carbon substrates which uses these specific parts of the PTS system. Specifically this comprises the proteins and mutants: PPA652 with a mutation in ptsG (or Enz IIBC^{glc}), PPA668 with a mutation in ptsM (or EnzIIAB^{man}) and PPA689 which is the double mutant, ptsG,ptsM (or EnzIIBC^{glc},EnzIIAB^{man}). All strains are described in Picon A., et al, Biotechnology and Bioengineering, vol. 90, No. 2, Apr. 20, 2005.

Vectors:

The vectors are all constructed to result in a variable rate of synthesis of the protein product.

Origin of replication: Two different origins of replication are used, i.e. these results in two different copy numbers of the vector. One low copy number plasmid giving about 20 copies and one high copy number plasmid giving 50-100 copies per cell. This allows for the variation of the total expression level in a cell.

Transcription: Two different promoters are used with the two different copy number origins. The lacUV5-promoter is used for low and high copy number vectors and the malK-promoter is used for low copy number vectors. The lacUV5-promoter is induced by adding isopropyl-β-D-galactopyranoside (IPTG, 5-300 μM) while the malK-promoter is dependent on cAMP and maltotriose formation for full induction. The initiation of the production may thus be varied by using these two different promoters which are characterised by different strength and different induction kinetics. Furthermore, it is possible to use different amounts of induction agent, which in turn results in different amounts of total protein and different ratios of soluble protein and inclusion bodies. The strains are all lacI which means that IPTG diffuses into the cell instead of being taken up actively through a protein transporter. This increases the possibility to achieve a distinct level of expression in relation to a distinct level of IPTG addition.

The promoters and their induction is the key to control of the synthesis rate and thus to the production of difficult proteins.

Translation: In fed-batch cultivation, the translation can be regulated by different substrate (glucose) feed rates or glucose uptake rates. If the cells are induced at a relatively high feed-rate (i.e. substrate uptake rate) there is generally more product produced than if you induce the cells at a low feed rate. On the other hand you risk higher degree of proteolysis and higher ratio of inclusion bodies. For many protein products productivity thus stands in direct opposition to the product quality.

Secretion: It is possible to introduce a signal peptide in order to transport the product to the periplasm. One such signal peptide is the signal peptide from Outer Membrane Protein A (OmpA). It has been found that the metabolic burden, with respect to overflow metabolism and the stress response named stringent response” is diminished if the product is transported to the periplasm.

Cultivation: By the mutations in the strains above, it is achieved a glucose regulation in the uptake which results in a restriction inside the cell. This means that the cells create their own fed-batch cultivation even though they are batch cultivated under excess conditions. This is a great practical advantage since batch cultivation is easier to perform but it is possible to reach higher cell densities and thus increased total production with fed-batch.

Temperature: Different temperatures may be used to vary the growth rate of the cells. If the cells grow slower all processes in the cells take longer time. This time can be used for correct folding of more difficult proteins.

Purification: The vectors have two types of purification tags, one histidine tag (His-tag) and one Zbasic-tag (Zb-tag). The His-tag is often comprised of six consecutive histidines; it is a small tag that usually does not disrupt the function, activity or structure of the protein. The usual way of purifying the His-tag is by IMAC (Immobilized metal affinity chromatography). IMAC uses the ability of histidine to bind to chelated metal ions, e.g. Cu²⁺, Zn²⁺ and Ni²⁺, with Ni²⁺ being most preferred. Zb is a synthetically produced version of the B-domain of Staphylococci protein A. Specific amino acids on the surface of this protein have been exchanged for positively charged amino acids so that the molecule may be more effectively captured by ion exchange chromatography on a cation exchange resin. The domain can be used at relatively high pH (7.5), where most E. coli proteins do not bind. This is generally the case for all proteins. The vectors may be used either with or without one or both tags.

Cleavage: The protein is cleaved from the purification tag by the protease 3C. This can be commercially purchased. Regardless of what system is used to produce the proteins, they will be flanked by a few extra amino acid residues. This is because proteases used to cleave a purification tag has a recognition sequence which might leave residual amino acids on the protein side. In this case this is 8 amino acids and the cleavage is between two of these. On the N-terminal side you get 2 extra amino acids (Gly and Pro) and on the C-terminal side you get 6 amino acids extra (Leu, Glu, Val, Leu, Phe, Gln). The exception is when you produce a protein without Zb-tag and without solubility reporter, where you get no extra C-terminal amino acids.

Reporter protein: In all vectors there is the possibility to produce a reporter protein on the C-terminal side of the protein of interest. In the cytoplasmic vectors Green Fluorescent Protein (GFP) is used as solubility reporter. GFP is a protein which is fluorescent when it has been expressed and correctly folded. By fusing GFP to the protein of interest it is possible to control that the protein has been produced and is in soluble form without the need for further analysis. GFP cannot be exported to the periplasm and therefore another reporter protein is present in the vectors with transport of the protein to the periplasm. This protein can either be β-lactamase or an alkaline phosphatase. Both may be analysed with a simple and commercially available assay.

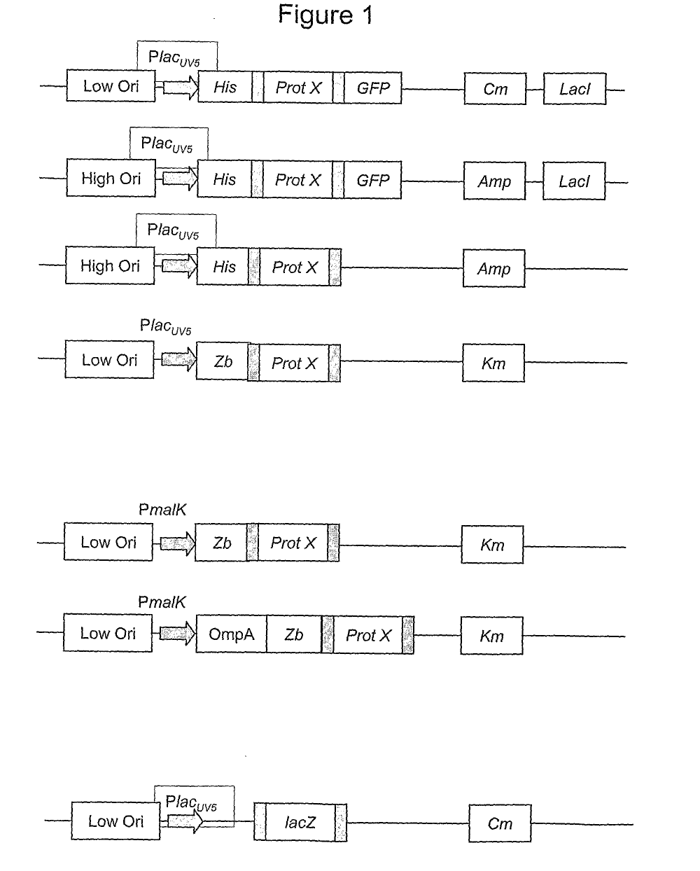

The design of the vectors is shown in FIG. 1.

The vectors are produced, amplified and introduced into the host strains according to procedure well-known to the person skilled in the art.

It is to be understood that the above description of the vectors only serve the purpose of explaining the invention by reference to specific examples. A person skilled in the art may readily design other vectors and strains adapted to his or her specific area of research. The full scope of the invention is that of the appended claims.

Experimental Section

### Example 1

Materials and Methods

Plasmid and Model Protein

To get a desired product protein a plasmid with suitable genes can be transformed into the bacteria. This particular example used the plasmid pAF1016 (P_{lacUV5}-lacZ, Cm^{R}) that has a lacUV5 promoter and produces the enzyme β-galactosidase. The plasmid was constructed by A. Farewell, Univ. of Gothenburg, for the EU Framework IV project “Control and optimisation of bottlenecks in recombinant protein production by Escherichia coli” or COOP. The recombinant protein produced was β-galactosidase.

β-Galactosidase

The E. coli enzyme β-galactosidase (5-gal) (encoded by the lacZ gene) is a tetramer consisting of four identical sub-units, each consisting of 1023 amino acid residues (Kalnins et al, 1983). It was chosen as the model protein because of its intracellular location, proteolytic stability, solubility at high concentrations, and ease of analysis. β-galactosidase hydrolyses lactose and other β-galactosides into monosaccharides in E. coli. It completely breaks down lactose into galactose and glucose, and it does so in two different ways. Firstly, it cleaves lactose into galactose and glucose. Secondly, it acts as a transglycosylase converting lactose into allolactose, and then it hydrolyses allolactose into galactose plus glucose. The enzyme is widely used as an reporter molecule in a variety of different assays.

The lacUV5 Promoter

The lac operon is a cluster of genes in E. coli consisting of structural genes (lacZ, lacY, lacA), regulatory gene, promoter and operator. This gene cluster functions coordinately in the induction of enzymes for the metabolism of lactose. Because all strains used in this project are lac⁻ we will be able to measure the β -gal production only produced by transcription of the plasmid gene. The lac repressor is a protein encoded by the lacI gene in the lac operon capable of binding with the lac operator and repressing the lac operon. Induction is caused by allolactose or IPTG (isopropylthiogalactoside) binding to the repressor. The strains used in this project are lacI⁻, so the expression of the coding gene for β-gal is constitutive. This means that it is expressed as a function of the interaction of RNA polymerase with the promoter without additional regulation (i. e. all the time), and we will not have to use an inducer for production start.

The lac promoter is the DNA region required for the initiation of transcription of the lac operon (Reznikoff and Abelson, 1980). The lac promoter can interact with RNA polymerase and the catabolite gene activator protein, CRP (also termed CAP). Transcription initiation is believed to occur by the following process. CRP in the presence of cyclic adenosine 3′,5′-monophosphate (cAMP) binds to the promoter. Binding of the CRP-cAMP complex stimulates the interaction of RNA polymerase with the promoter, and a lac mRNA transcript can be initiated.

The promoter has been defined by three classes of point mutations and by several deletions. The class-I and class-II point mutations are cis-dominant mutations, which decrease the lac operon expression level. Class-I mutations decrease the response of the lac promoter to the presence of the CRP-cAMP complex (the CRP-cAMP complex has a lower affinity for the lac promoters that contain class-I mutations). The class-II mutations are believed to act by depressing the ability of the RNA polymerase to interact with the lac promoter, and some also alter the response to the CRP-cAMP complex. The class-III mutations facilitate lac expression in the absence of the CRP-cAMP complex. The mutants have been constructed by isolating Lac⁺ mutants in a crp⁻ (lacking CRP) and/or cya⁻ (lacking adenyl cyclase) background, or by isolating second-site revertants of class-I mutations. These mutations are thought to act by facilitating the interaction of RNA polymerase with the lac promoter.

The lacUV5 promoter is a derivative of the lac promoter, and it carries two point mutations in the CRP-binding site rendering the expression insensitive to catabolite repression. The UV5 mutations belong to the class-III mutations and they map between the deletion L8 and the operator. The mutations enhances CRP-cAMP-independent lac transcription, e.g. the promoter will not require cAMP to be fully activated and can thus be activated under excess of glucose by IPTG, which binds to the repressor and cause a conformational change of the repressor that can no longer bind to the operator. The RNA polymerase is free to bind the operator and start transcription.

Bacterial Strains

Isogenic strains defective in the enzyme PtsG (IICB^{Glc}), the enzyme PtsM (IIAB^{Man}) or both transport systems were constructed in the EU Framework IV project COOP (Picon et al, 2005). The strain PPA652 is defective in the PtsG, the strain PPA668 is defective in the PtsM, and the strain PPA689 is defective in both PtsG and PtsM. The strains were constructed from the wild-type strain AF1000 (MC4100, relA⁺, lac⁻) via P1-transduction.

Strain PPA652 (Glc-defective, ptsG) was made by replacing the ptsG gene by a ΔptsG::Km cassette from PPA290 (ΔptsG::Km). Strain PPA668 (Man-PTS defective, ptsM) was constructed in a two-step process. In the first step, strain WA2127 (ΔmanX) was the recipient of a P1-lysate from strain CAG12074 (zea-3068::Tn10). Selection was performed on tetracycline-containing plates. In the second step, AF1000 was the recipient of a P1-lysate from the previously selected strain. Strain PPA689 (ptsG and ptsM) was derived from PPA668 by using a P1-lysate from PPA290. The strains used are shown in Table 1.

TABLE 1

| | | Source or |
|---|---|---|
| STRAIN | Relevant characteristics | reference |
| AF1000 | MC4100, relA⁺, wild-type | A. Farewell |
| PPA668 | AF1000, ΔmanX, zea-3068::Tn10 | A. Picon |
| PPA652 | AF1000, ΔptsG::Km | A. Picon |
| PPA689 | PPA668, ΔptsG::Km | A. Picon |
| PPA290 | ΔptsG::Km | A. Picon |
| WA2127 | ΔmanX | A. Picon |
| CAG12074 | zea-3068::Tn10 | A. Picon |
| AF1000 + | MC4100, relA⁺, P_{lacUV5}-lacZ, Cm^{R} | combination used |
| pAF1016 | | in this work |
| PPA668 + | AF1000, ΔmanX, zea-3068::Tn10, | combination used |
| pAF1016 | P_{lacUV5}-lacZ, Cm^{R} | in this work |
| PPA652 + | AF1000, ΔptsG::Km, | combination used |
| pAF1016 | P_{lacUV5}-lacZ, Cm^{R} | in this work |
| PPA689 + | PPA668, ΔptsG::Km, | combination used |
| pAF1016 | P_{lacUV5}-lacZ, Cm^{R} | in this work |

Cultivation Media

Minimal medium consisted of: 7.0 g of (NH₄)₂SO₄, 1.6 g of KH₂PO₄, 6.6 g of Na₂HPO₄.2H₂O and 0.5 g of (NH₄)₂—H-Citrat per litre of deionised water. 1 ml of trace elements solution and 1 ml of 1M Mg SO₄ was sterile-filtrated through a Minisart 0.20 μm Sartorius syringe filter to one litre of medium. Trace element solution consisted of (g per litre): CaCl₂*2H₂O, 0.5; FeCl₃*6H₂O, 16.7; ZnSO₄*7H₂O, 0.18; CuSO₄*5H₂O, 0.16; MnSO₄*4H₂O, 0.15; CoCl₂*6H₂O, 0.18; Na-EDTA, 20. Yeast medium consisted of Bacto™ Yeast Extract, Difco, from Becton Dickinson, USA and was diluted according to their prescription. D-glucose and D-maltose were autoclaved separately and added to the media. The D-glucose was added to a concentration of 5 gram per litre in all pre-cultivation shake flasks, and a concentration of 10 gram per litre in all batch cultivations and shake flask experiments, if nothing else is recounted. Solid medium contained 23 gram per litre of Difco™ Nutrient Agar from Becton Dickinson, USA. When needed antibiotics were added to the media: tetracycline (25 mg/l), kanamycine (50 mg/l) and chloramfenicol (34 mg/l).

The same minimal media and the same cultivation conditions were used in batch cultivations both in fermenter and in shake flask experiments if nothing else is recounted. All shake flask experiments were performed in one litre shake flasks with total liquid volume of 0.1 litre, except from the pre-cultivation shake flasks that were done in 5 litre shake flasks with total liquid volume of 0.5 litre. For addition of amino acids during shake flask experiments an Amino acid standard solution from Sigma was used. The standard contained all amino acids except for the amino acids glutamine, asparagine and tryptophan, which were weighted and diluted in deionised water to a suitable concentration (g/l: glutamine 0.37, asparagine 0.33, tryptophan 0.51) before addition to the medium. 2.5 μM of all the amino acids was added to the medium before cultivation start. The amino acids were sterile-filtrated through a Minisart 0.20 μm Sartorius syringe filter into the medium. Adding 1 gram per litre of D-glucose and 3.6 gram per litre of D-maltose to the shake flasks did the glucose/maltose experiments, and the control flasks contained only 10 gram per litre of D-glucose.

Growth Conditions and Genetic Methods

Cells were thawed in room temperature from −80° C. frozen stocks and directly diluted into the culture medium. Cultures were grown in a HT Infors Minitron® Incubator Shaker at 37° C. at 180 rpm. The bioreactor was sterilised together with the medium at 121° C. for 20 minutes before cultivations. The glucose was sterilised separately. For batch cultivations, 0.5 litre cell suspension was grown to an OD(600) of 2 before it was inoculated into the bioreactor (Belach AB, Sweden, total volume 14 litre) to a total volume of 8 litre. Batch cultivations were performed at 37° C. in minimal media at 100-800 rpm with a start glucose concentration of 10 g/l. To register the dissolved oxygen level (DOT) a polarographic oxygen electrode was used and the oxygen level never dropped below 30% air saturation. The pH was kept at 7.0 by addition of 25% ammonia solution. Breox was used as the antifoam agent. Samples for analytical determinations were withdrawn during the cultivations. In the cAMP shake flask experiment 4 mM of cAMP was added when the cells had reached an optical density at 600 nm (OD(600)) of 0.6. The yeast shake flask experiment was done as follows: Strains PPA652 and PPA689 were grown in yeast medium. After reaching an OD(600) of 0.8 for PPA652 and 0.5 for PPA689, 18 ml of cell suspension was taken out from both flasks respectively. The 18 ml was centrifuged in a Sorvall® RC26 PLUS Superspeed Centrifuge at 2500 rpm, 4° C. for 15 min, thereafter the supernatant was discarded and the cell pellet was diluted in a shake flask containing minimal medium. The yeast shake flasks were also allowed to continue growing. After about 15 hours measurements were done again. Transformations of plasmids into the cells were performed by electroporation with a Bio-Rad Micro Pulser™.

Analyses

Optical density at 600 nm (OD(600)) of samples from shake flask cultivations and fermenter cultivations was measured by a Novaspec® II Visible spectrophotometer from Amersham Pharmacia Biotech. During shake flask and fermenter cultivations with bacteria with the plasmid 5 ml samples for activity measurements were collected. The samples were disintegrated in a FRENCH® pressure cell press from SLM AMINCO®, SLM instruments. Thereafter the β-galactosidase activity was measured as the turn over rate of the substrate o-nitro-phenyl-β-D-galactopyranoside (ONPG) per ml of solution. It was done by measuring the absorbance at 410 nm at 25° C. in an Ultrospec 3000 UV/Visible Spectrophotometer from Amersham Pharmacia Biotech (see method used in Sandén et al, 2002). Samples were kept on ice during times before and after disintegration. Disintegrated samples were also put on a gradient activity gel; 4-12% Tris-Glycine Gel from Invitrogen™ life technologies, Novex®. A Tris-Glycine running buffer and a Native Tris Gly Sample Buffer were used, and the gels were run at 120 V for 180 min. The running buffer consisted of 25 mM Tris Base, 192 mM glycine and deionised water. In batch cultivations, biomass concentration was determined as cell dry weight (g/l). The triplicate dry weight samples were usually withdrawn every 30 min. The 5 ml samples were put in pre-dried and pre-weighted glass tubes and centrifuged at 4° C. at 4500 rpm for 10 min in a Biofuge primoR Heraeus centrifuge from Kendro, the supernatant was discarded, the pellet was resuspended in 5 ml deionised water and centrifuged the same way again. Thereafter the supernatant was discarded and the glass tubes with the pellet were dried in a Thermocenter, Salvis, Swissmade oven overnight at 120° C., before they were weighted again. Samples from batch cultivations for acetic acid (HAc) concentration determination were withdrawn at the same times as for the dry weight determinations, by taking the supernatant of the dry weight samples after their first centrifugation. The supernatant was sterile-filtrated through a Minisart 0.20 μm Sartorius syringe filter and frozen at −20° C. before analysis. Later the concentrations of the thawed HAc samples were analysed on a Labsystems iEMS Reader MF spectrophotometer with an enzyme kit from Boehringer-Mannheim, cat. No. 14826. The glucose amount was measured by Combure-Test® E glucose sticks from Roche during the shake flask experiment. Glucose samples from batch cultivations were withdrawn usually every hour during cultivation, and every ten minutes during the last hour of cultivation. A 2 ml sample of cell suspension was diluted in 2 ml 0.132 M perchloric acid (to inactivate substrate uptake) and centrifuged at 4° C. at 4500 rpm for 10 min in a Biofuge primoR Heraeus centrifuge from Kendro. 3.5 ml of supernatant was put in a new tube and mixed with 75 μl 3.6 M K₂CO₃. The samples were frozen at −20° C. until analysis. Later the concentrations of the thawed glucose samples were analysed on a Labsystems iEMS Reader MF spectrophotometer with a D-Glucose/D-Fructose enzymatic Bioanalysis UV-test from Boehringer Mannheim. If the concentration of glucose in a sample were higher than 0.5 g/l the concentration was measured by another UV method instead (see appendix for details on UV method and calculations). Samples for Viable count determination were withdrawn at an OD(600) of 2, 5 and 10 during cultivations with cells including the plasmid. The cell suspension was spread on Nutrient Agar plates with and without the appropriate antibiotics in concentrations of 5·10⁻⁶, 5·10⁻⁷ and 5·10⁻⁸ gram per litre.

Results

Determination of Growth Rate, Acetic acid Formation, β-Gal Production and Substrate Consumption.

Batch cultivations with bacteria without the plasmid were performed with the three mutants, PPA668 (ptsM), PPA652 (ptsG) and PPA689 (ptsG,ptsM), and also with the wildtype strain AF1000 in order to determine what effect the plasmid itself would have on the four parameters. Batch cultivations with bacteria including plasmid were performed with ptsM and the wildtype strain. The two remaining mutants ptsG and ptsG,ptsM both bearing the IICB^{Glc} mutation did not show sufficient β-gal production in minimal medium, so that further experiments with them came to focus on finding a way to make them start producing the protein. Those experiments were performed as shake flask cultivations and includes the use of complex media combinations. Samples from bioreactor cultivations were taken to optical density measurements, biomass concentration, acetic acid concentration, activity measurements, glucose consumption and viable count determinations.

Growth Rate Determination

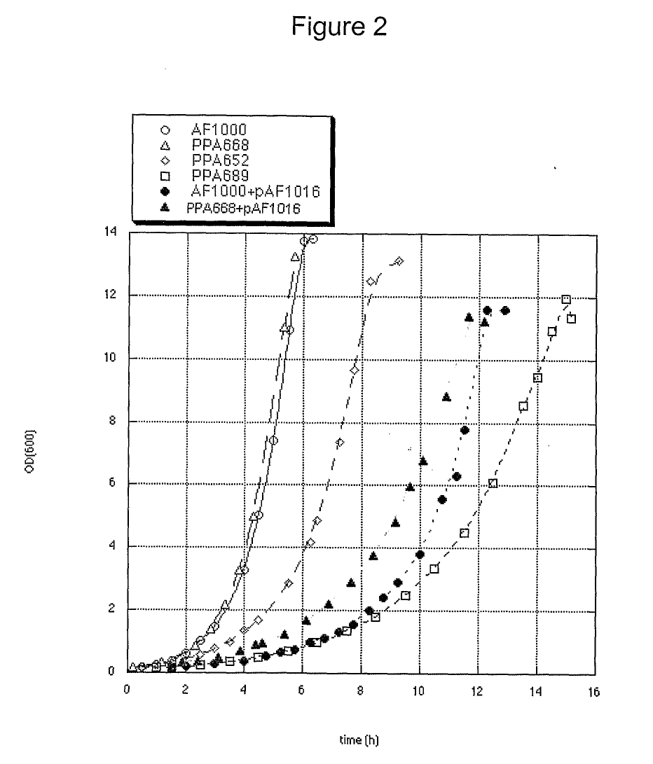

The effect of the restricted uptake rate in batch cultivation on the growth rate is shown from OD measurements in FIG. 2. The measurements showed that ptsM reached about the same cell density per ml as the wildtype strain at about the same time; an OD(600) of 13-14 was reached after six hours. PtsG grew slower and reached an OD(600) of 13 after nine hours, and the double mutant ptsG,ptsM reached an OD(600) of 12 after 15 hours. The cultivations with the wildtype strain and ptsM including the plasmid took twice as long time as the cultivations without plasmid, and reached only an OD(600) of 11.5. PtsM grew somewhat faster than the wildtype strain both with and without the plasmid, despite the IIAB^{Man} mutation.

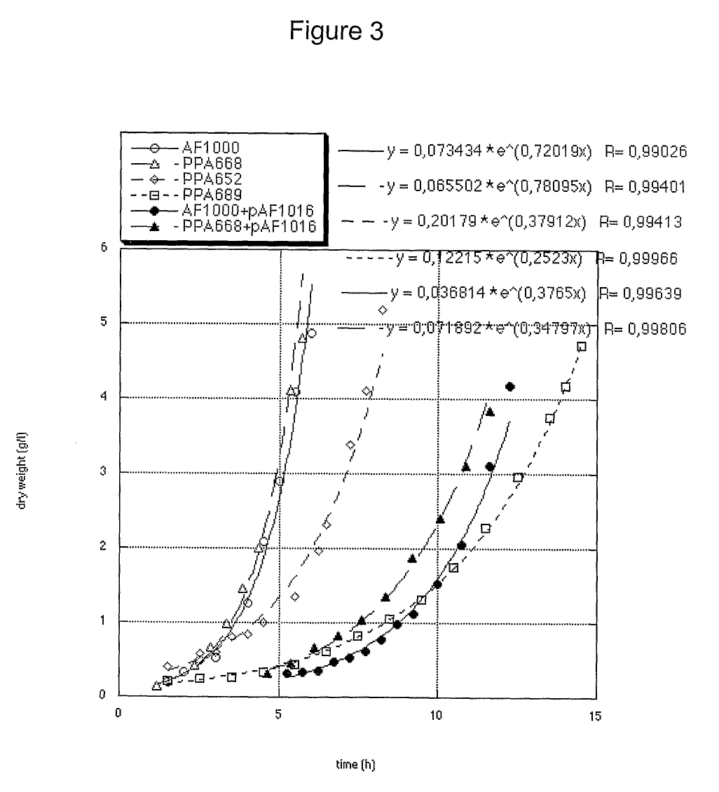

The dry weight determinations showed the same pattern as the OD measurements according to the cultivation time and the biomass concentration that was reached. When the OD(600) was plotted against the dry weight this gave a linear relationship (data not shown). The dry weight results from all six batch cultivations can be seen in FIG. 3. It was seen that the data fitted the exponential equation y=k*e^{μ}^{ₓ}, where y is the dry weight per litre at the time x, k is a constant and μ is the specific growth rate. Therefore exponential curve fits have been fitted to the data in the graph. The different strains showed the following growth rates: wildtype μ=0.72, ptsM=0.78, ptsG μ=0.38, ptsG,ptsM μ=0.25, wildtype+pAF1016 μ=0.38 and ptsM+pAF1016 μ=0.35. Calculations showed that the errors in the dry weight determinations causes an error E in the specific growth rates that was E<±0.02. In other words, the difference of 0.06 between the wildtype and ptsM is significant. The burden of the plasmid in the wildtype and ptsM approximately reduced the growth rate by half. The equations y=k*e^{μ}^{ₓ} for each strain can be seen in Table 2.

The conclusion is that although the medium contains high amounts of glucose this is not taken up by the cells and the mutations work in a comparative fashion to the fedbatch technique performance.

TABLE 2

| Strain | Equation, y = k*e^{μ}^{ₓ} | μ (h⁻¹) |
|---|---|---|
| AF1000 (wildtype) | y = 0.073*e^{0.72x} | 0.72 |
| ptsM | y = 0.066*e^{0.78x} | 0.78 |
| ptsG | y = 0.202*e^{0.38x} | 0.38 |
| ptsM&ptsG | y = 0.122*e^{0.25x} | 0.25 |
| wildtype + pAF1016 | y = 0.037*e^{0.38x} | 0.38 |
| ptsM + pAF1016 | y = 0.072*e^{0.35x} | 0.35 |

β-Galactosidase Production

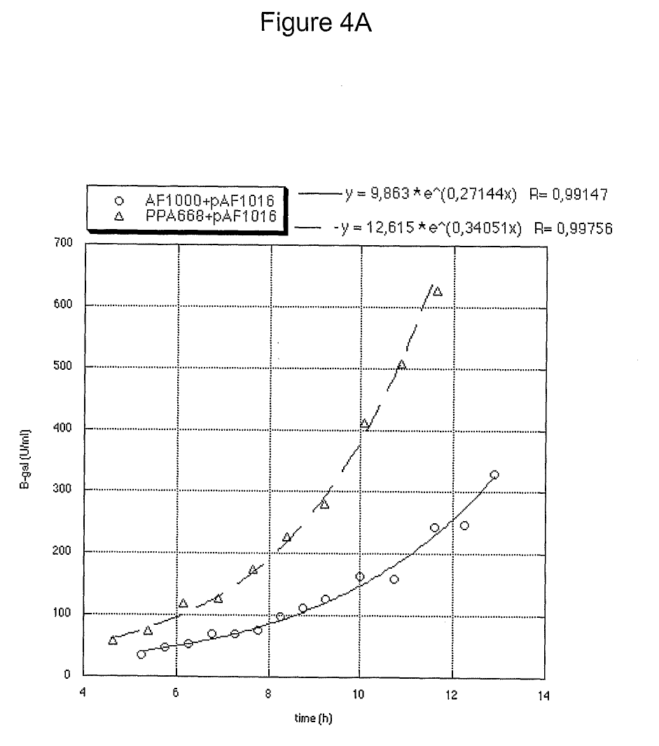

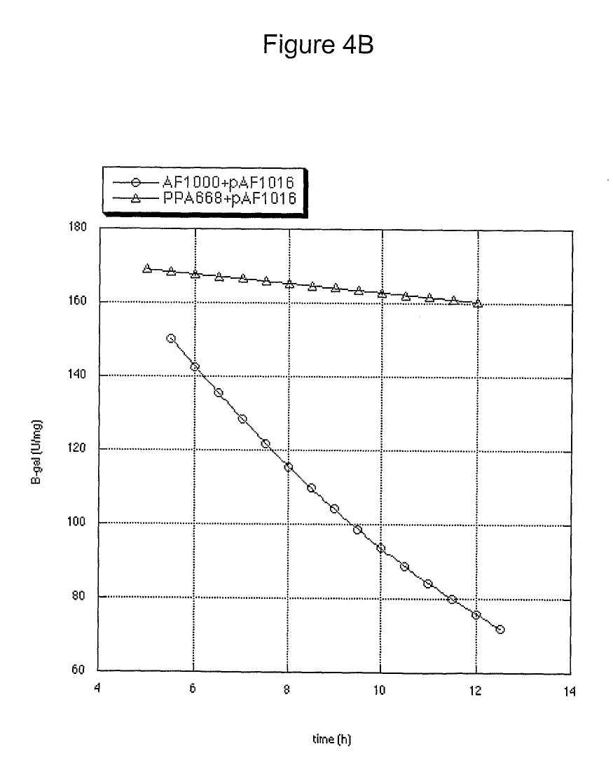

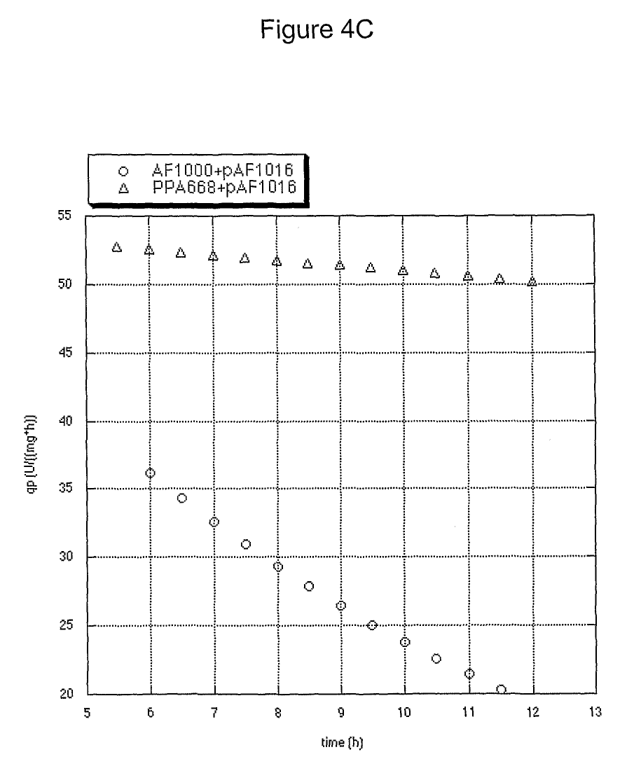

Analyses of the β-gal activity are shown in FIGS. 4A, B and C from cultivations in minimal medium. The time course of production of β-gal as the volumetric accumulation is shown in FIG. 4A. The production of β-gal is exponential which can be seen in this figure. Curve fits have been done by the equation y=k*e^{qx} where y is the β-gal activity in Units/ml at the time x, k is a constant and q is the production rate. PtsM grew faster and reached a higher protein activity level in a shorter time than the wildtype did. The β-gal concentration in U/mg dry weight, FIG. 4B, shows that the β-gal accumulation per cell is higher in ptsM than in the wildtype. FIG. 4C show the specific production rate qₚ in U/(mg*h) derived from the β-gal and dry weight curve fits. qₚ is higher in ptsM than in the wildtype strain, and it is also almost retained unchanged throughout the cultivation in ptsM. By looking at the figures, it seems like if cultivation had continued the protein production level would have continued to stay high.

Calculations showed that in the end (t=12.5 h) of the batch cultivation with the wildtype strain the protein concentration was 0.37 mg/ml and that corresponds to 19% of the total protein in the cell. In the end (t=11.5 h) of the batch cultivation with ptsM the protein concentration was 0.79 mg/ml and that corresponds to 40% of the total protein. Surprisingly, the mutant PtsM, produces much faster than the wild type in spite of high acetic acid production. The activity gels of the β-gal protein showed three bands (data not shown). The bands in the bottom are the β-gal tetramer consisting of four identical 135 kDa subunits. The two other bands consist of eight subunits and twelve subunits respectively and are thus multimeres of the protein. The Viable Count determinations in batch cultivations showed that the plasmid was retained in the bacteria throughout the full cultivation time.

Acetic Acid Formation

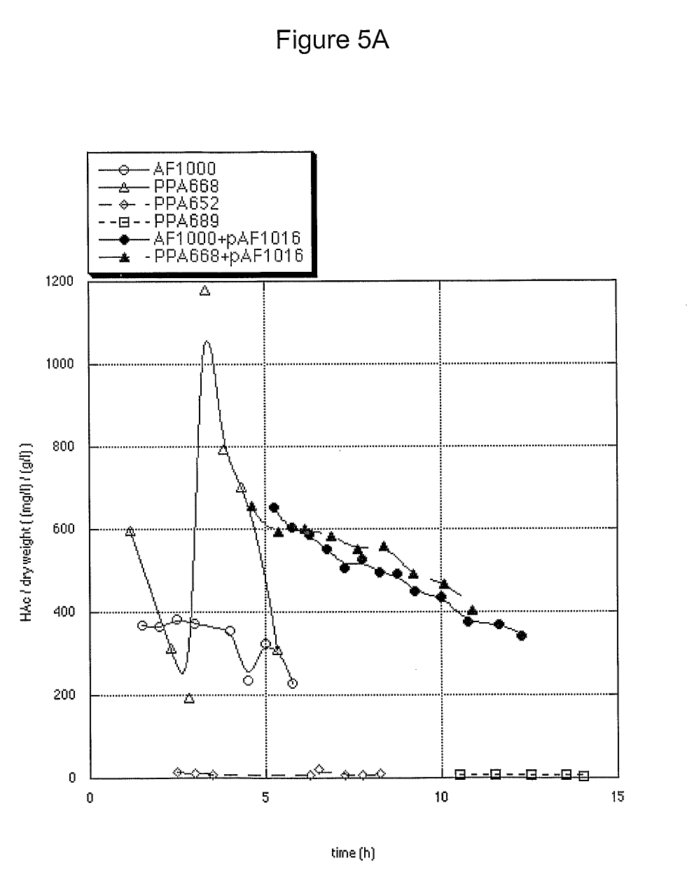

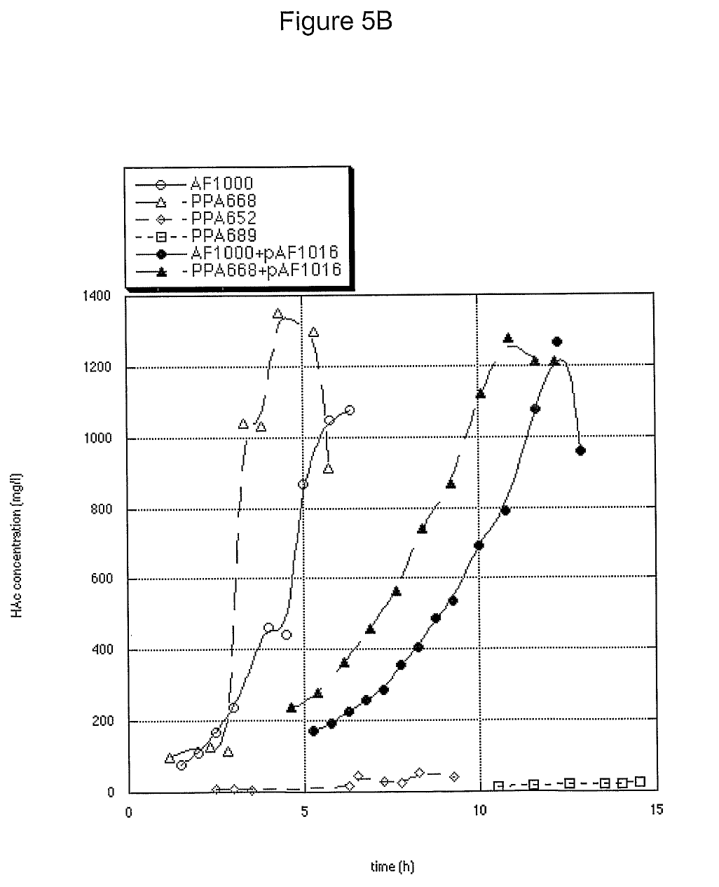

In FIG. 5 the acetic acid accumulation for the six bioreactor cultivations is shown. The wildtype strain and ptsM cultivations show a high acetic acid production both with and without the plasmid, whereas ptsG and ptsG,ptsM show a very low production. The higher production in the wildtype and ptsM without the plasmid is a result of the higher substrate uptake rate in these cultivations, as is the lower production in the ptsG-mutated strains is due to their lower uptake rate, all in spite of very high concentrations in the medium. PtsM reach about the same apex level of acetic acid production during both cultivations, and the wildtype strain increase its apex production level with about 2 g/l when bearing the plasmid. In FIG. 5B the acetic acid concentration/dry weight is plotted against time. The main trend here is that the acetic acid concentration decreases as opposed to dry weight. PtsM show a very varying quotient. The conclusion is that overflow production can be avoided in batch cultivation in the mutants ptsG and the double mutant.

Substrate Consumption

The specific substrate consumption rate qₛ was determined for all six batch cultivations. The uptake rates correspond to the growth rates.

Investigation of Effects of Stringent Response and CCR on Induction of the ptsG Mutant Strains

For two of the mutants i.e. those defective in the ptsG, production could not be achieved in minimal medium to the extent that it was clearly visible. However, in yeast extract medium production was clearly visible. Since one initial experiment showed that a transition from yeast extract to minimal medium indeed could be used to induce the lacUV5 promoter the hypothesis was that this might be caused by the change in substrate either by evoking of a) the stringent response or b) by a cAMP accumulation. The stringent response and the raising of CCR were thus used to try to manage production. The stringent response is a phenomenon of reduction in synthesis of protein and RNA caused by the deprivation of an essential amino acid. It is accompanied with accumulation of the alarmone ppGpp (guanosine tetraphosphate) and is a way of signalling stress in the cell. ppGpp inhibits transcription of some genes, because it is necessary for the cell to shut down some synthesis when suddenly it is deprived of a single or a few amino acids. However, ppGpp also appears to activate transcription of other genes (Lengeler et al, 1999). The lacZ gene coding for β-gal is reported to be under a positive control during the stringent response, which means that the stress of suddenly being deprived of an amino acid could start the production of β-gal. In order to manipulate the stringent response cells were subjected to intended deprivation of amino acids. To test the effect of CCR the cells were subjected to an external addition of cAMP or to cAMP formation by growth in a medium with two substrates both taken up via the PTS.

To test the different courses of action the ptsG-mutants were first cultivated in a nutrient-rich yeast medium and from there suddenly put in a minimal medium. This gave production of about 250 U/ml at an OD(600)of 8 in yeast flasks but did not start the production in minimal medium flasks. Cells were then cultivated in shake flasks in minimal medium with addition of amino acids to try the same hypothesis but because the amino acids were not allowed to end during cultivation time it is hard to tell if the hypothesis worked here. Shake flask experiments were also done with the aim to raise the CCR, to see if this would start the production. Shake flasks that contained two carbon sources, glucose and maltose were cultivated. When the glucose ends in the medium, the CCR is raised when the cell is trying to adapt to the new carbon source. The cAMP level increases which might have a possible effect on the lacUV5 promoter, so the hypothesis was that this might start the production of β-gal. A cAMP-test was also done; The lacUV5 promoter is said to be cAMP independent. To test the hypothesis that the lacUV5 promoter is dependent on cAMP while the Glc-mutation cause the cAMP levels to decrease, a cAMP shake flask experiment was done. In this experiment a large addition of cAMP was added to see if this would start up the production.

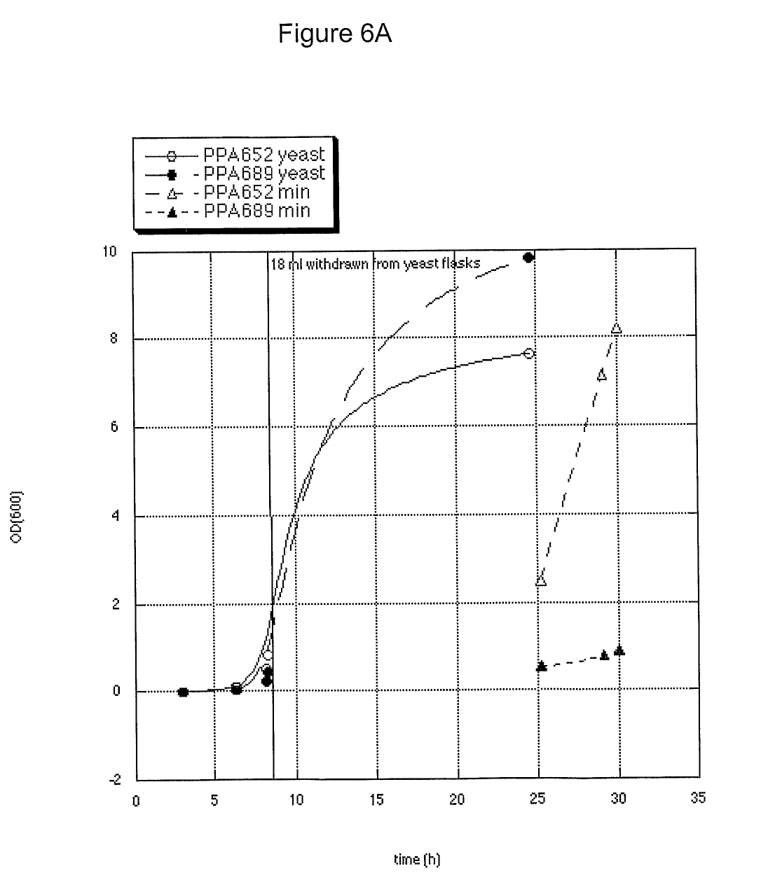

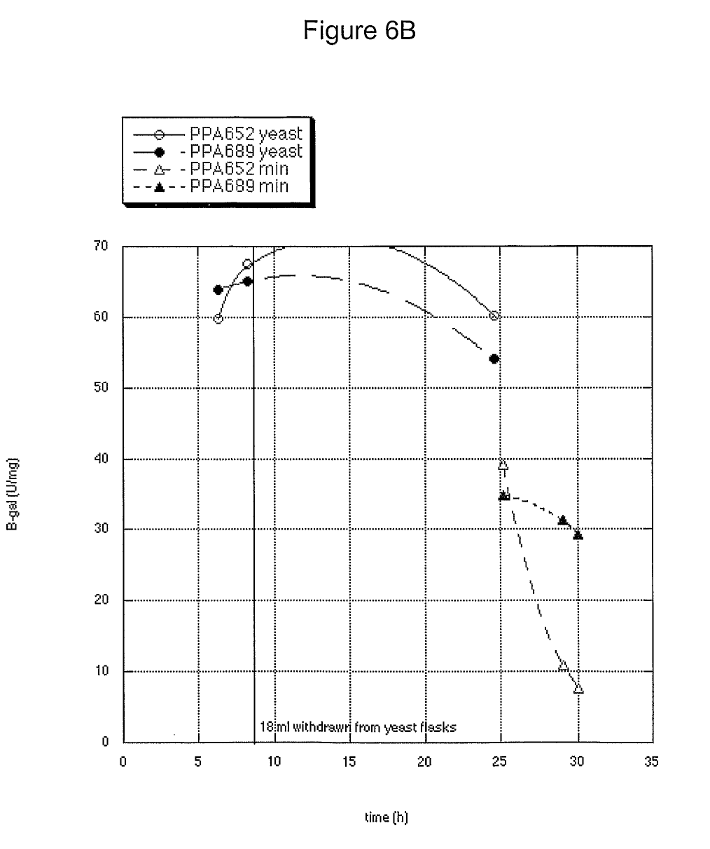

Using yeast medium instead of minimal medium did the yeast experiments. OD measurements can be seen in FIG. 6A. In the yeast flasks after about 25 hours ptsG had reached an OD(600) of about 7.5 and ptsG&ptsM had reached an even higher OD(600) at about 10. In the minimal medium on the other hand both strains seems to have had a very long lag period, but quite fast after that ptsG started to grow, much faster than ptsG&ptsM. The β-gal activity in U/mg for the shake flasks is shown in FIG. 6B. The activity seems to be decreasing in both yeast flasks and minimal medium flasks. Both yeast flasks and minimal medium flasks seems to have their apex production level somewhere before t=25 but where is hard to decide.

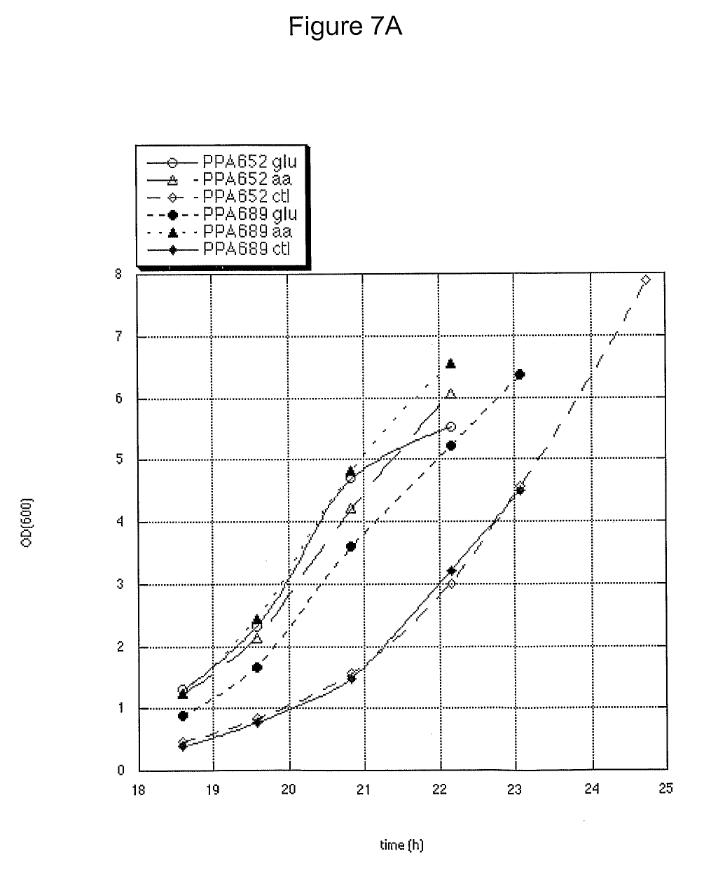

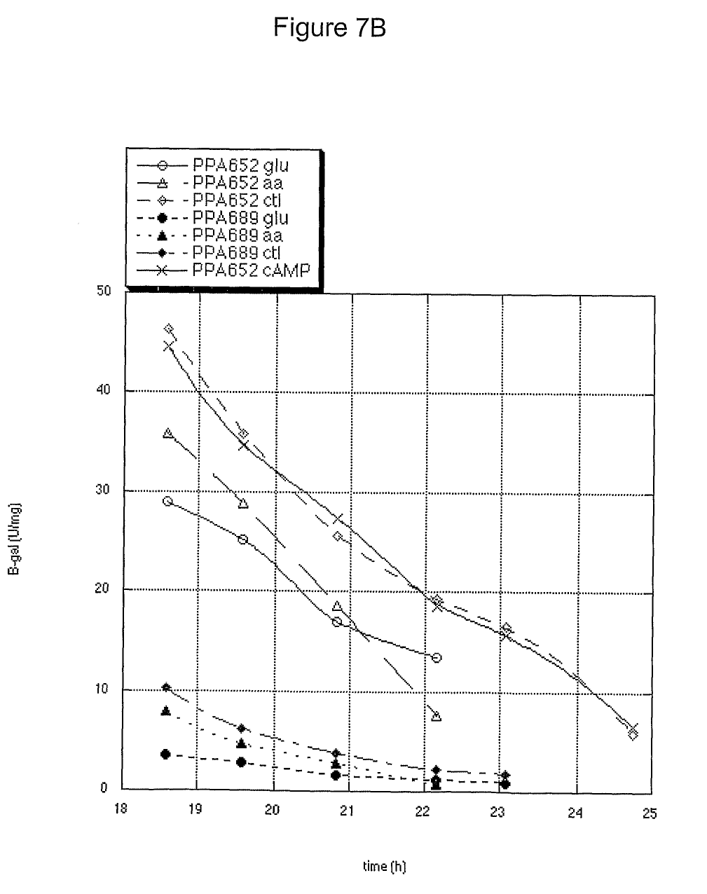

In the amino acid experiment, the addition of the amino acids to the medium made these cells grow faster than the controls as expected, which can be seen in FIG. 7A. The β-gal activity in U/mg is shown in FIG. 7B. The activity is about the same as in the controls at the same cell density and the activity seems to be decreasing with the same rate in both amino shake flasks and controls. Because the amino acids were not allowed to end during the cultivation it is hard to tell if the stringent response of amino acid deprivation would start the production here.

In the glucose/maltose experiments both glucose and maltose were present in the medium from cultivation start as opposed to the controls which only contained glucose. With the yield coefficient 0.5 kg/kg (amount of cells produced per kg of consumed substrate) the glucose supply should end around an OD(600) of 1 (if we suppose that an OD(600) of 2 corresponds to a cell concentration of 1 g cells per litre). The sticks showed that glucose was present in the medium with ptsG to an OD(600) of at least 2.5, and with ptsG,ptsM to an OD(600) of at least 5, so the carbon catabolite repression does not seem to be working here and we do not get the diauxic growth that we expected. Both strains with maltose in the media grew faster than the controls, which can be seen in FIG. 7A. The β-gal activity in U/mg is the same as in the controls at the same OD(600) and also seems to be decreasing with the same rate as can be seen in FIG. 7B, so this test did not seem to start production of β-gal.

In the cAMP experiment (which was only performed with ptsG) the cAMP was added when the cells had reached an OD(600) of 0.6, directly after the first sample collection at time t=18.58. The β-gal activity in U/mg is shown in FIG. 7B, and seems to be exactly the same as in the control both before and after the cAMP addition. These results show that addition of cAMP does not start any production, so that the lacUV5 promoter is probably cAMP independent.

The protein production is slightly higher in ptsG than in ptsG&ptsM in all cultivations (amino acids, glucose/maltose, controls) at the same optical cell densities which can be seen in FIG. 7B, which might be due to the larger stress on this strain because of the two mutations. The reason that the β-gal activity per mg is decreasing in all shake flask experiments seems to be due to a dilution of the protein, so the protein production must have stopped at some time during the cultivations. The antibiotics contained in the media and earlier Viable count determinations have shown that the plasmid is still present in the cells, so probably the plasmid is still there and there is another explanation to why the production still ends.

Discussion

Determination of Growth Rate, Acetic Acid Formation, β-Gal Production and Substrate Consumption.

Growth Rate

The growth of all strains was exponential, and the growth rate was found to be strongly reduced in the strains lacking functional PtsG. The growth rate of ptsM (μ=0.78), exceeded that of the wildtype (μ=0.72) and it was also somewhat higher than found in earlier research (μ=0.69) (Picon et al, 2005). (The same minimal medium was used except from the medium used here contained 7 g/l of (NH₄)₂SO₄ instead of 2 g/l as used in Picon et al). Calculations of the error in the measurements showed that the difference in growth rate between AF1000 and ptsM is significant. The difference could be due to an up-regulation of ptsG transporters while the ptsM is absent. The wildtype and ptsG showed growth rates almost the same as found earlier (wildtype now μ=0.72, earlier μ=0.69, and ptsG now μ=0.38, earlier μ=0.46). The double mutant ptsG,ptsM (μ=0.25) had a slightly higher growth rate than showed in earlier research (μ=0.13). The higher growth rate in ptsG than in ptsG,ptsM confirms that a non-functioning ptsG takes help from ptsM in transporting glucose (Picon et al, 2005). Bearing the low copy plasmid pAF1016 further reduced (approximately halved) the growth rate which is due to the burden of copying the plasmid.

Acetic Acid Formation

The acetic acid formation is due to a situation when catabolism is exceeding the anabolism in the cell. The wildtype and ptsM strains produced much more acetic acid in the batch cultivations (both with and without plasmid) than the PtsG mutated strains. The lower acetic acid formation in the PtsG mutated strains was obtained, most certainly because of the lower substrate uptake rate in these strains. These PtsG mutated strains had the lower by-product formation that we hoped for—they have the same effect as the common fed-batch on acetic acid accumulation and thereby fulfill the demanded criteria. The cultivations including plasmid also had a low growth rate, still they produced up to 1.2 g/l of acetic acid. This is understandable if they still consume glucose at the same rate as without the plasmid. The glucose consumption rate was determined by an enzymatic analysis, which at high glucose concentrations demanded many dilutions, which resulted in multiplication of errors causing unclear results. Due to the unclear substrate consumption results, it is uncertain how they affected the acetic acid accumulation. The acetic acid formation in the cultivations with plasmid could also be due to the effects that the β-gal production has on the metabolism, however by an unknown regulation.

β-Galactosidase Production

PtsM reached a higher protein production level of β-gal than the wildtype strain. This could be due to the higher growth rate in ptsM. The specific production rate was also higher in ptsM and it stayed at almost the same level throughout the whole cultivation, which is unusual to not say unmatched by data for all available production systems today used. Earlier research has shown that the wildtype in fed-batch with an inducible promoter show a high specific production rate for about four hours before the production quickly decreases (Sandén et al, 2004). Here the specific production rate stays on approximately the same level for about seven hours and shown no sign of declination. The amount of β-gal of the total protein in the cell was calculated to be 40% which is a high value compared to earlier research results done with wildtype in fed-batch that show values of about 30% (Sandén et al, 2004).

Conclusions

A summary of the obtained results is shown in Table 4, which shows the change in the parameters for the strains compared to the wildtype in a batch cultivation. The growth rate and the by-product formation rate are decreased in the two ptsG strains. To that extent the demanded criteria are fulfilled. On the other hand, those two strains do not produce any protein to a clearly visible extent in minimal medium. The ptsM strain showed a slight increase in all three parameters, growth rate, specific production rate and by-product formation rate. One reason for these results of the three mutant strains could be that different stock collections for the same strain with the plasmid can show different protein production results, which was also seen during cultivations. Another explanation to the received results could be an unknown regulation, involving the regulatory protein Mlc and might be due to the combination of the deletion in the host strain together resulting in its affect of the control of the promoter P_{lacUV5}. The regulatory protein Mlc (encoded by the mlc gene) represses, among others, the ptsHI, ptsG and malT genes. The nonphosphorylated form of the glucose PTS enzyme two B, IIB^{Glc}, (=high ptsG level) binds to the Mlc protein, thus relieving the repression. If ptsM is absent, this will give an upregulation of the ptsG transporters, the four parameters (growth rate, acetic acid formation rate, protein production rate, glucose consumption rate) will increase and the nonphosporylated IIB^{Glc} will bind to Mlc which would give a decreased repression of the genes. The question is, does mlc also repress the lac promoter? If that is the way, an absent ptsM would give a decreased repression of the lac operon and the result would be more protein production. If on the other hand ptsG is absent that would give no raise of the repression and therefore lower protein production. To be able to understand this regulation, measurements of for example the phosphorylation state of IIB^{Glc} in the different strains has to be done. The phosphorylation state of the glucose PTS enzyme two A, IIA^{Glc}, has been determined in the strains (A Picon et al, 2005) when glucose is present in the medium, where it was mainly in its unphosphorylated form in strains the wildtype strain, ptsM and ptsG. In strain ptsG,ptsM the IIA^{Glc} was mainly in its phosphorylated form. Also, it has been shown that when glucose is absent from the culture medium, both IIA^{Glc} and IIB^{Glc} will be mainly in their phosphorylated state in the wildtype (Gosset, 2005). A possible explanation to the results could be that mlc represses the lac operon, but further research on the phosphorylated state of IIB^{Glc} in the mutated strains must be done to show if the hypothesis is true.

TABLE 4
Growth, byproduct and product formation in fedbatch cultivation
with the WT compared to batch cultivation with the mutants.

| | wildtype | ptsM | ptsG | ptsG, ptsM |
|---|---|---|---|---|
| Parameter | in fedbatch | in batch | in batch | in batch |
| growth rate | [symbol] | [symbol] ? | [symbol] | [symbol] |
| acetic acid | [symbol] | [symbol] ? | [symbol] | [symbol] |
| formation rate | | | | |
| protein production | [symbol] | [symbol] ? | no prod. | no prod. |
| rate(minimal medium) | | | | |
| protein production | [symbol] | no data | [symbol] | [symbol] |
| rate(minimal medium) | | | | |

Investigation of Effects of Stringent Response and CCR on Production Rate of the Mutant Strains

The yeast shake flask experiment showed high levels of protein production also for the tho mutants PtsG and PtsGPtsM, which indicated that something in the yeast medium made the cells able to produce the protein in spite of their ptsG-mutation. It could just be the simple explanation that in the minimal media the cell focuses all its energy on growing. And in the yeast media all nutrients makes the cell not having to synthesise that many substances itself, so that it can also produce the protein without a lot of extra effort. After 25 hours of cultivation, when the yeast flasks as well as ptsG in minimal media had reached a high OD(600) (8 or higher) and ptsGptsM in minimal media had reached an OD(600) of 1, the β-gal activity per mg seemed to be decreasing for all shake flasks because of dilution with growth (the cells had at some point stopped producing the protein but continued to grow and accumulate in cell mass). In conclusion, activation of the stringent response in this way did not manage production in the minimal medium shake flasks.

The amino acid shake flasks grew faster than the controls as expected, because not having to make the amino acids themselves makes growing easier for the cell. To test the hypothesis of that the stringent response can start production the test would have to be done with lower acetic acid concentration to make them deplete during cultivation. In a nutrient rich medium, as in the yeast flask experiment, a higher cell density gives a higher protein production. This is not the case in minimal medium, where the production seems to decrease when the cell density is increasing, whether there are amino acids present or not.

In the glucose/maltose shake flask experiment both strains grew faster than the controls. This means that somehow the carbon catabolite repression is not working in these cells. It has been shown earlier that mutants in the PTS system can be able to make use of more than just the glucose carbon source at the same time (Gosset, 2005). The cells in this experiment can utilize the maltose from the beginning and before the glucose is depleted in the media. This was also indicated by the measurements with the glucose measurement sticks, which showed that glucose was still present in the medium with ptsG to an OD(600) of at least 2.5, and with ptsGptsM to an OD(600) of at least 5. Earlier research has found that IIA^{Glc} is mostly in its non-phosphorylated form in ptsG when glucose is present in the medium (Picon et al, 2005). Maybe this is not the case here and the phosphorylated IIA^{Glc} can activate adenylate cyclase (cAMP levels increase) to be able to take in the maltose at the same time as the increased Man-PTS transport can translocate glucose over the membrane. Or maybe more likely the IIA^{Glc} is mostly in its non-phosphorylated form but the inducer exclusion is not working as usual. In ptsG&ptsM on the contrary, IIA^{Glc} has been found earlier to be mostly in its phosphorylated form (Picon et al, 2005), and this might create higher adenylate cyclase and cAMP than usual which could cause faster uptake of the maltose and cause the higher growth rate. The glucose/maltose flasks show levels of β-gal production similar to that of the controls compared at the same OD(600). In the cAMP shake flask experiment where a large addition (4 mM) of cAMP was added to see if this would start up the production, the addition of cAMP did not start up the production of β-gal. In conclusion, the promoter is believably cAMP independent, and the cAMP does not seem to play any conspicuous part in these shake flask experiments.

### Example 2

Materials and Methods

Strains and Vectors

Escherichia coli, AF1000 was used in all experiments (Sandén A M et al, 2002). Three mutations were used which are all situated in the genes for expression of the phosphoenolpyruvate:carbohydrate phosphotransferase system (PTS) and results in a reduced uptake rate of glucose (Picon A et al, 2005). The three strains are defect in either the enzymes IICB^{Glc} (ptsG), IIAB^{Man} (manX) or both. These strains will hereafter be referred to as PTS_{Glc}, PTS_{Man} and PTS_{GlcMan}, respectively. The construction of the present strains was described elsewhere (Sandén A M et al, 2002, Picon A et al, 2005). All strains and plasmids are listed in Table 5.

TABLE 5
Summary of Escherichia coli strains
and plasmids used in this example.

| | | Source/ |
|---|---|---|
| Strain/plasmid | Relevant characteristics | reference |
| AF1000 | MC4100, relA⁺, wild-type | A. Farewell |
| PPA668 | AF1000, ΔmanX, zea3068::Tn10 | A. Picon |
| PPA652 | AF1000, ΔptsG::Km | A. Picon |
| PPA689 | PPA668, ΔptsG::Km | A. Picon |
| F′ | lacI_{q}, lacY, lacA, Tn^{R} | A. Farewell |
| pAF1016 | P_{lacUV5}-lacZ, Cm^{R} | A. Farewell |
| | | (Unpublished) |
| pBCP622 | Pₐᵣₐ-ptsG, Amp^{R} | A. Picon |
| | | (Unpublished) |

Tn = tetracycline, Km = kanamycin, Cm = chloramphenicol, Amp = ampicillin

The product protein was β-galactosidase. The protein was produced either constitutively (lacI_{q}) or induced from the lacUV5 promoter. Plasmid pAF1016, originally constructed from pTL61T containing the lac operon but where the natural promoter was replaced with lacUV5 to gain insensitivity to catabolite repression (Linn T et al, 1990), and pACYC184 (Rose R, 1998), was used. From these vectors the amp^{R} gene was further removed. This vector includes the origin of replication from pACYC184, which was chosen to introduce a low copy number and the chloramphenicol resistance gene rendering the plasmid compatible with the mutant strains. The repression of the lacUV5-promoter was controlled from an F′-factor (lacI_{q}, lacY, lacA, Tn^{R}). The constitutive production system was equal in all aspects to the inducible except from the use of the F′ episome with the repressor, which was omitted. The plasmid pBCP622 holds the ptsG gene under the control of the inducible arabinose promoter. The plasmid contains the gene for ampicillin resistance and the pBluescript replicon. This plasmid was in some experiments used simultaneously to pAF1016.

Cultivation Medium

A minimal salt medium was used which consisted of (per litre): 7 g (NH₄)₂SO₄; 1.6 g KH₂PO₄; 6.6 g Na₂HPO₄*2H₂O; 0.5 g (NH₄)₂—H-Citrate. The medium was autoclaved at 121° C. in the bioreactors and supplemented with 1 ml l⁻¹ 1 M MgSO₄ and 1 ml l⁻¹ trace element solution, which was sterile filtered (Sartorius 0.2 μm) into the bioreactors. Composition of the trace element solution was (per litre): 0.5 g CaCl₂*2H₂O; 16.7 g FeCl₃*6H₂O; 0.18 g ZnSO₄*7H₂O; 0.16 g CuSO₄*5H₂O; 0.15 g MnSO₄*4H₂O; 0.18 g CoCl₂*6H₂O; 20.1 g Na-EDTA. In the case of high cell density cultivation these additions were repeated whenever the optical density (OD, measured at 600 nm) was increased by a factor of 20.

Glucose, which was autoclaved separately was added as carbon source to a concentration of 5 and 10 g l⁻¹ in shake flask and batch bioreactor cultivations, respectively. In a specific batch experiment in bioreactor, repeated additions of 10 g l⁻¹ glucose was added when the initial batch glucose was exhausted to maximise the cell mass accumulation. In the fed-batch cultivations the initial glucose concentration was 2.3 g l⁻¹, except for experiments with inducible expression where the feed was started immediately i.e. without an initial batch phase.

For cultivations using the production vector, 34 mg l⁻¹ chloramphenicol was added. For the experiment with the mutant PTS_{GlcMan} grown in yeast medium, 5 g l⁻¹ yeast extract was added to the minimal medium prior to sterilization but the MgSO₄ and trace elements solutions were in this case excluded.

The glucose feed concentrations when no product was produced were 400 g l⁻¹ (giving a theoretical growth of 0.25 h⁻¹) and 600 g l⁻¹ (giving a theoretical growth of 0.38 h⁻¹). During induced expression the feed was divided into three stages, the first feed concentration was 65 g l⁻¹, the second 200 g l⁻¹ and the third 600 g l⁻¹. The different feed concentrations were used to match the corresponding pump speed ranges.

Cultivation Conditions

One-litre shake flasks were cultivated with a working volume of 100 ml at 37° C. in an incubated shaker at 180 rpm (Infors Minitron). The medium was inoculated with 1.5, 2 ml frozen PTS_{Glc} cells (OD=0.8) and PTS_{GlcMan} cells, respectively. Five litre shake flask cultures with a working volume of 500 ml were used to inoculate the bioreactors. An amount of 62.5 ml l⁻¹ of the exponentially grown cell suspension with an OD of 2 was used for inoculation of the batch bioreactors.

The wild type fed-batch cultivations without production were inoculated with 0.4 ml l⁻¹ of a frozen stock solution stored in −80° C., which was added directly to the bioreactor (same as above) after sterilization and addition of all medium components.

The fed-batch cultivations were started after a batch phase on glucose with the exception of the cultivation with the wild type strain producing the product, where the feed was started directly after inoculation. When glucose was consumed the oxygen signal was used to start the exponential feed. This feed was set to give the theoretical growth rates of 0.38 h⁻¹ and 0.25 h⁻¹, respectively, which was designed to correlate to the maximum growth rates of the mutants PTS_{Glc} and PTS_{GlcMan}, respectively.

The feed profiles of the fed-batch cultivations were calculated from mass balances for exponential feed and of the limiting substrate, glucose, according to:

F(t)=F₀*e^{μ}t

where F₀ is given from:

F₀=(μ*V*X)/(Sᵢ*Y_{X/S})

where F (1 h⁻¹) is the feed rate at a specific time event t (h), F₀ is the initial feed rate at fed-batch start (1 h⁻¹), μis the specific growth rate (h⁻¹), V is the cultivation volume (l), X the cell dry weight (g l-⁻¹) and Y_{X/S} the theoretical yield coefficient based on the quotient of the cell mass production rate over the substrate uptake rate (taken as 0.5 g g⁻¹).

The experiments were carried out either in an 8-l bioreactor with a working volume of 5.0 l (Belach Bioteknik AB) or a 12-l bioreactor with a working volume of 8.0 l (Belach Bioteknik AB). Temperature was set to 37° C.±0.1° C. and pH was kept at 7±0.1 by addition of ammonia (25% w/w). The bioreactor was equipped with a polarographic electrode to monitor the dissolved oxygen tension (DOT). The liquid phase oxygen was controlled by manually changing the stirring speed (200-1200 rpm) or the gas flow into the bioreactor (1-121 min⁻¹). DOT was thus kept above 20% in all cultivations. In cultivations where the production plasmid pAF1016 was induced, induction took place at an OD of 1 with 300 μM of IPTG. An antifoam agent, Breox, was added when necessary.

Analyses

Biomass

Cell growth was followed by measurement of light scattering at 600 nm in a Novaspec II Spectrophotometer (Amersham Pharmacia Biotech AB). The data are presented as OD. The cell suspension was always diluted by 0.9% (w/w) sodium chloride to OD=0.1 before analysis. Biomass concentration (DW) was determined as cell dry weight (g l⁻¹) by centrifugation (10 min, 4500 rpm) of 3*5 ml cell suspension in pre-dried and pre-weighed test tubes in a Wifug Lab centrifuge after which the pellet was collected and dried overnight at 105° C. before weighing. At exponential growth the correlation between these variables was approximately OD=2.5*DW.

Acetate

The supernatant from the biomass determination was sterile filtered (Sartorius, 0.2 μm) and used for acetate analysis. The acetate concentration was analyzed by a commercial enzymatic kit (Boehringer-Mannheim no. 148261). Each sample was analyzed four times from which the mean value was calculated.

Glucose

Samples for glucose determination were taken as described previously (Larsson G et al, 1996), where rapid inactivation of the substrate uptake is achieved by sampling (under <0.1 s) into test tubes containing cold (+8° C.) perchloric acid (0.132 M). This was followed by neutralization by 3.6 M K₂CO₃ and centrifugation at 4500 rpm for 10 minutes (Biofuge primo R, Heraeus) before the samples were analyzed using a commercial enzymatic kit (Boehringer-Mannheim, kit no 716251). Each sample was analyzed four times and a mean value was calculated.

β-Galactosidase

The product, β-galactosidase, is produced in the cytoplasm of E. coli and the analysis is based on activity measurement of this enzyme. To disrupt the cell, 10 ml cell suspension was disintegrated in a high-pressure homogeniser (SLM Aminco, using a French Pressure Cell FA-073). Cell disintegrate, 100 μl, was mixed in a cuvette with 2.7 ml assay buffer and 300 μl of the enzyme substrate o-nitrophenyl-β-D-galactopyranoside (ONPG). If the absorbance reached above 3 absorption units, the samples were diluted with assay buffer to stay below this limit. The assay buffer consisted of: 0.2 M Na-phosphate (pH7.0), 2 mM MgCl₂, 8% methanol and 0.25% Tween 20. The enzyme activity was analyzed photometrically at 410 nm and 25° C. by measuring the increase of the absorbance during one minute (Ultrospec 3000, Pharmacia Biotech). The output, i.e. the enzymatic activity (U ml⁻¹), is given from the equation dA/dt*(ε*L)⁻¹ where dA/dt is the change in absorbance per minute, ε is the extinction coefficient which under these conditions is 2.1 abs.units*cm²*μmol⁻¹ and L the cuvette length of one cm. One unit (U) corresponds to the hydrolysis of one μmol ONPG min⁻¹. From earlier quantification experiments the amount of one mg of β-galactosidase is known to correspond to approx. 800 U. Each sample was analyzed two times and a mean value was calculated.

Results

In fed-batch cultivation a restriction in the feed of a chosen substrate to the bioreactor, most often glucose, leads to a restricted uptake of the same substrate. This, in turn, reduces the growth rate as well as the respiration and heat evolution. Additionally, the production of overflow metabolites, such as acetic acid, is reduced. This allows the accumulation of cells to high density and the harmful effects of acetic acid on some products, specifically recombinant proteins, are aborted. These are highly desirable effects in production wherefore most industrial processes are run in fed-batch mode.

However, in small-scale multi-parallel reactor systems the control of a fed-batch is difficult, if not impossible, and this reduces the possibility to achieve the positive effects gained by fed-batch technology. This is a serious drawback when the task is to produce a large number of different proteins of unknown structure and function.

The aim of this example was to find a solution to overcome this limitation and the strategy was to choose a set of strains which by directed mutagenesis were lacking parts of the phosphoenol pyruvate:phosphotransferase system (PTS). This would in practice lead to a restricted uptake of glucose. This restriction is therefore done at cell rather than reactor level. Glucose is taken up over the cytoplasmic membrane mainly through the specific glucose uptake system or the mannose uptake system, which are dominated by the proteins II^{Glc} or II^{Man}, respectively. At very low glucose concentrations also GalP and the Mgl-system becomes important (Gosset G, 2005) but they were not considered of importance under the present experimental conditions. The set of strains used thus reflected these primary enzymes and had mutations in either or both. The strains are referred to as: PTS_{Man}, PTS_{Glc} and PTS_{GlcMan}, respectively, where the subscript denotes the mutation. The hypothesis was that these strains would be culturable on a batch medium, including high concentrations of glucose, but that the uptake would be controlled by the composition of the permeases in each strain resulting in a maximum but restricted individual glucose uptake for each of them. If this was the case, the performance of the mutants in batch should be equal to the performance of the wild type (WT) cultivated at the same growth rate in fed-batch mode.

Cell Growth

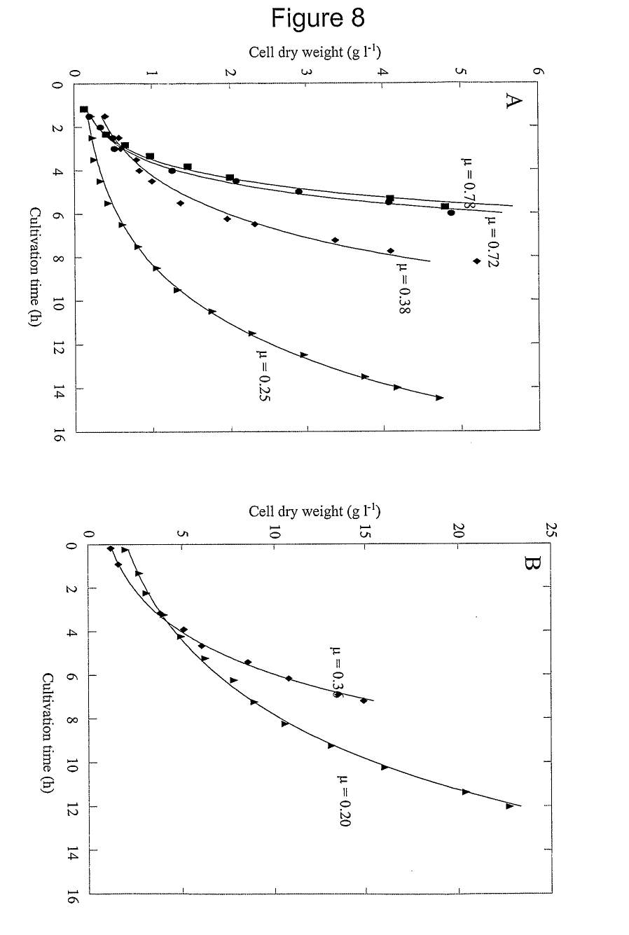

The first parameter evaluated was the growth rate. The three mutants were grown under batch cultivation conditions without product formation and the result is shown in FIG. 8a. The WT strain is included in the figure for comparison.

All mutant strains are growing exponentially to a cell density of approximately 5 g/L. This indicates an exponential uptake of glucose in spite of the deletions. The resulting growth rates are 0.78 h⁻, 0.38 h⁻¹ and 0.25 h⁻¹ for PTS_{Man}, PTS_{Glc} and PTS_{GlcMan}, respectively. The WT strain resulted in a maximum growth rate of 0.72 h⁻¹ but this cannot be considered to deviate from the PTS_{Man} strain. This shows that under concentrations of 10 g l⁻¹ glucose supply, there is no effect of the mannose deletion with respect to growth. The general conclusion is thus that despite the abundance of glucose it is possible to arrive at a growth limitation on the cell level and thus a lowering of the growth rate.

From the mutant growth data fed-batch cultivations with the WT cell was designed. An exponential glucose feed rate was chosen at a rate which would theoretically give the same glucose uptake rate as with the mutants. For the PTS_{Man} mutant the growth rate was already maximal wherefore no fed-batch experiment was possible to perform. The resulting growth pattern for the WT growth in fed-batch was also exponential, as expected, and the resulting growth rates were 0.36 and 0.20 h⁻¹, respectively as compared to 0.38 h⁻¹ and 0.25 h⁻¹ for the mutants. The fed-batch growth data are included in FIG. 8b.

Acetic Acid Production

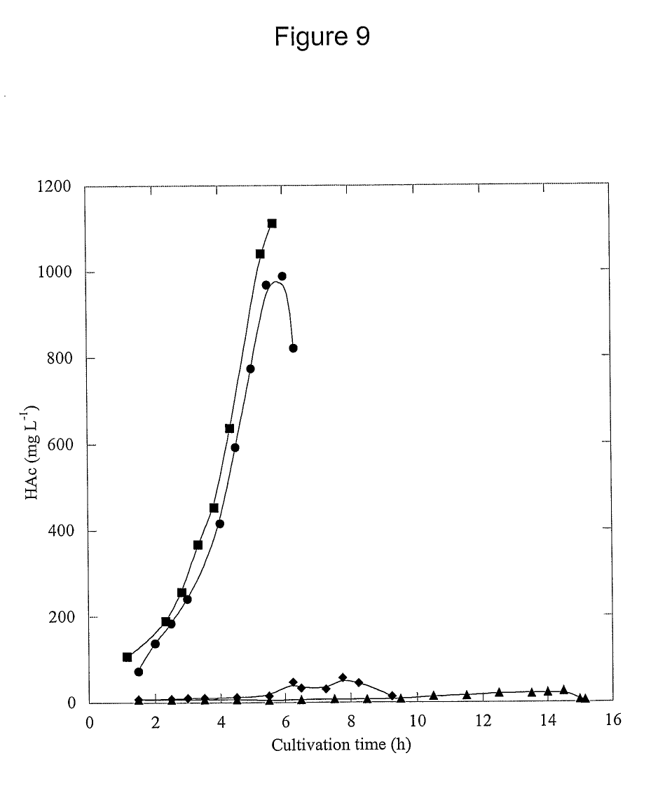

E. coli, grown on high glucose concentrations, is subjected to overflow metabolism (Doelle H et al, 1982). The availability of glucose leading to growth rates over approximately 0.35 h⁻¹ on minimal medium, pH 7 and 37° C. seems to be a switch-point (Meyer H-P et al, 1984). The reason is that the cell takes up glucose although it cannot be fully oxidised and this results in the production of mixed acids although the oxygen uptake rate is very high. The main metabolite formed is acetic acid, which was also analysed and the data are plotted in FIG. 9.

As can be expected the WT cell produces acetic acid in proportion to the growth rate. This is also the case for PTS_{Man}, which grows at the same rate as the WT, and it seems thus probable that the uptake rate of glucose is the same. For these two strains, the acetate concentration rapidly increases to over 1 g l⁻¹. The last sampling points taken during the WT cultivation indicate the event when acetate is being consumed due to glucose limitation. In large contrast, PTS_{Glc} and PTS_{GlcMan} do not produce large quantities of acetic acid. The concentration is thus close to zero for the larger part of the cultivations.

The comparison to fed-batch processing of the WT, at the same approximate feed rates shows that after the initial batch period where the glucose is consumed, these cells do not produce acetic acid due to the lowered feed rate as expected (data not shown). The reason for the late increase in acetic acid in the fedbatch (FIG. 9) is not known but is not likely due to oxygen limitation since the DOT was 16-19% and no effects on respiration was seen. The conclusion is that also with respect to acetic acid formation the batch processed mutants are equal to fed-batch processed WT cells.

Specific Oxygen Consumption Rate

Glucose entering a cell can theoretically be thought of as used for four different purposes in recombinant protein producing cells: for cell growth, for by-product formation, for maintenance and respiration and for product formation. So far, we can conclude that with respect to growth and by-products the WT and the mutants behave the same. However, due to the mutations it could be likely that the loss of the vital permeases could result in an increased respiration in order for the cell to gain more energy. The fact that the cells do not produce acetic acid does not mean that carbon is not taken up and used for this purpose. This would be a considerable drawback since these cells would be high consumers of oxygen and thus the total consumption of the population would hinder the accumulation of cells to high density.

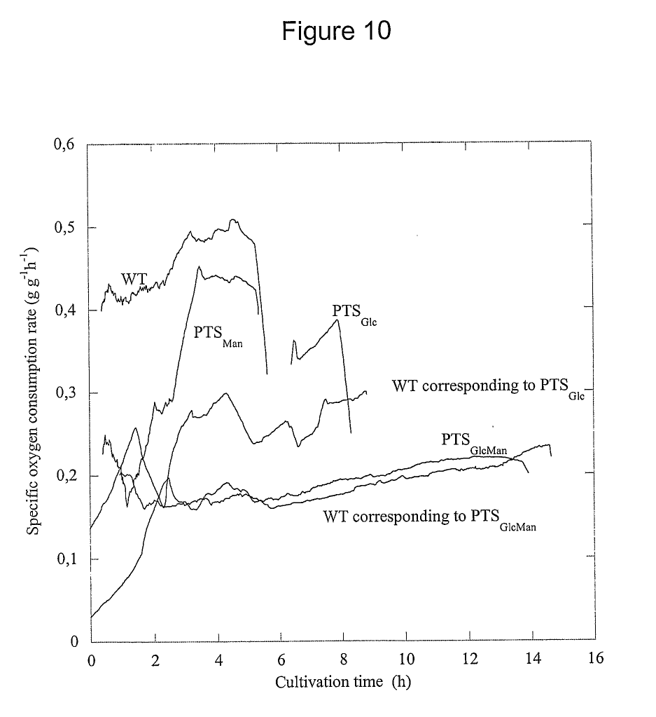

The fast growing cells i.e. the WT and the mutant PTS_{Man} have however the highest specific oxygen consumption levels (g g⁻¹h⁻¹) as shown in FIG. 10. In this case we observe that the mutant, PTS_{Man}, has a lower oxygen consumption rate than the WT, which thus shows an effect of the mutation that was not seen from growth and by-product formation data. Furthermore, the specific oxygen consumption rate increases with growth rate, which means that PTS_{Glc} has a higher consumption rate than the double mutant but both these mutant strains lies well below the WT values. The carbon dioxide production values are comparative over the time course of the cultivation (data not shown) why also the respiration rate of all the mutants is lower than the WT.

The comparison of mutants and WT at the corresponding growth rates achieved by fed-batch, shows that the oxygen consumption rate for the batch grown PTS_{GlcMan} cell is almost identical to the WT cultivated in fed-batch. From the respiration point-of view, this strain should thus be able to cultivate to the same biomass. The other slow growing mutant, PTS_{Glc}, has for unknown reasons, a slightly higher oxygen consumption rate than the corresponding fed-batch grown WT.

Product Formation

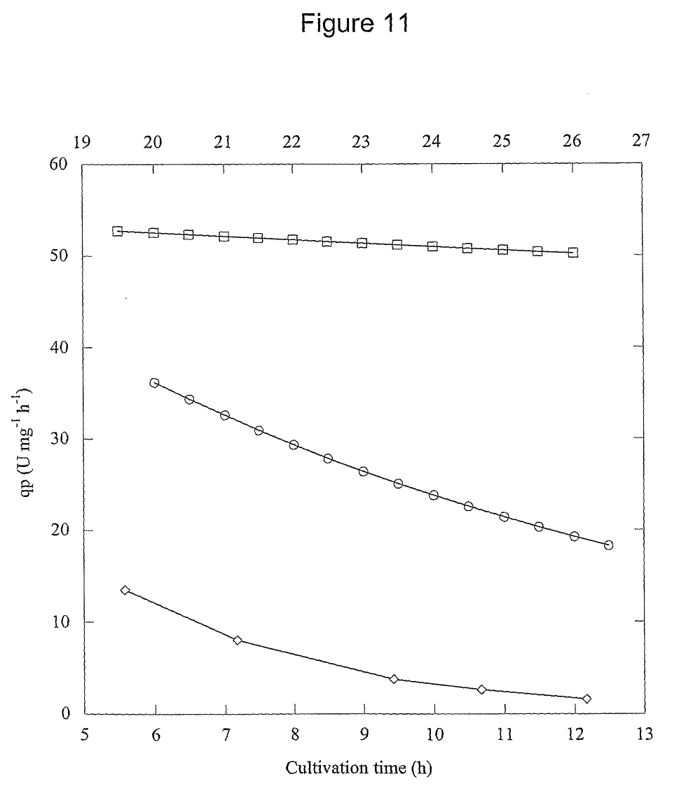

The product formation capacity of the mutant strains was evaluated with recombinant production of β-galactosidase as a model product protein. The expression was initially based on constitutive production from the lacUV5-promoter. The constitutive system was chosen to avoid the difficulty in setting a comparative time of induction in the two production systems (batch, fed-batch) and to eliminate the effect this could have on the comparison. In FIG. 11, the production data are shown in cultivations of WT and the mutants. It should be observed that these strains all grow at different growth rates since they are all batch cultivations. The highest and most stable specific production rate, i.e. 53 U mg⁻¹h⁻¹ was reached with PTS_{Man}, which also had the highest growth rate on minimal medium. This production rate is higher than in any system tested by us before for this product. The WT strain has also a relative high specific productivity, 36 U mg⁻¹h⁻¹, but the stability in production rate is drastically reduced some time into the cultivation and the rate has dropped by 50% at the point of harvest. It was likely to expect that the specific production rate should be constant when a constitutive production system was used. So is not the case and especially not for the WT. Further the two strains have approximately the same growth rate but very different specific production rate which means that not only the growth rate influences the production but also some factor arriving from the mutation.

The two mutants with the PTS_{Glc} mutation were principally only productive in cultivations with supplements of yeast extracts (data not shown). In the figure is plotted both the batch data of PTS_{Glc} on minimal medium (lowest values) and the high production rate levels (up to 63 U mg⁻¹h⁻¹) with PTS_{GlcMan} in batch cultivation with yeast extract supplement. However, even if the yeast supplement restores the production this also increases the growth rate, which is not acceptable under the constraints of this work. From additional experiments we tried to elucidate which was the production-triggering component of the yeast extract. This component was not found but we could conclude that it was neither the addition of amino acids nor the addition of cyclic AMP (cAMP). Of the coupling of growth and production can conclusively only be said that the higher the growth rate the higher was the initial production rate.

To evaluate if the low production rate of the PTS_{Glc} mutants was in any way arriving from the constitutive expression system we constructed an inducible system based on the lacUV5 promoter under otherwise equal conditions. We compared the expression in minimal medium from the WT with the PTS_{Glc} strain where the WT was cultivated in fed-batch mode and PTS_{Glc} in batch mode in order to induce the cultures at the same growth rate, 0.4 h⁻¹. After induction the specific growth rate decreased to 0.15 h⁻¹ and 0.20 h⁻¹ for the WT and PTS_{Glc} respectively.

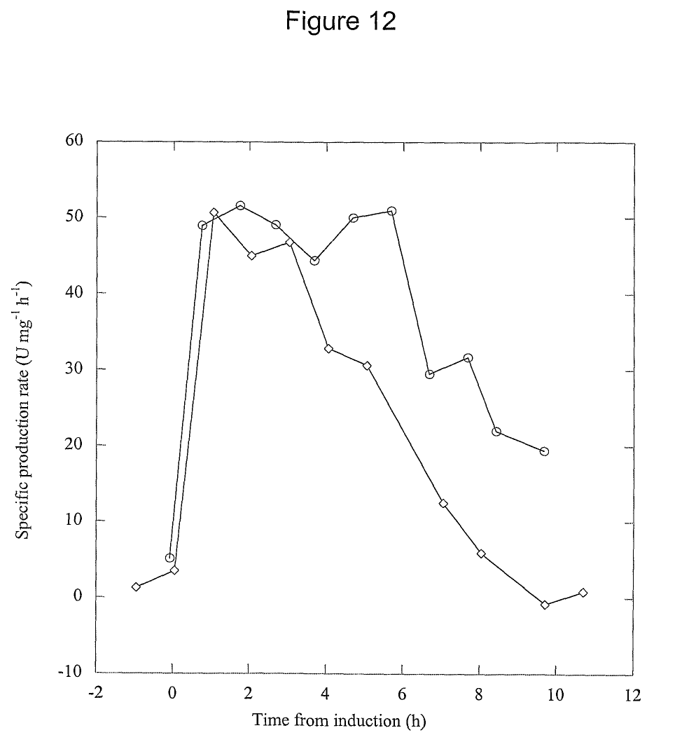

Both strains were shown to rapidly accumulate the product but with the highest rate immediately after induction. The result is shown in FIG. 12. The specific product formation rate showed a maximum of approximately 50 U mg⁻¹h⁻¹. As for the constitutive expression, the WT strain drops in production rate to 50% of the initial value, which takes place after approximately six hours after induction. For the PTS_{Glc} strain, this rate could be maintained for three hours before rapidly diminishing to some 20% of the initial value. The total β-galactosidase accumulation at the end of the cultivation estimated to 33% and 13% of the total protein, respectively.

High Cell Density Cultivation

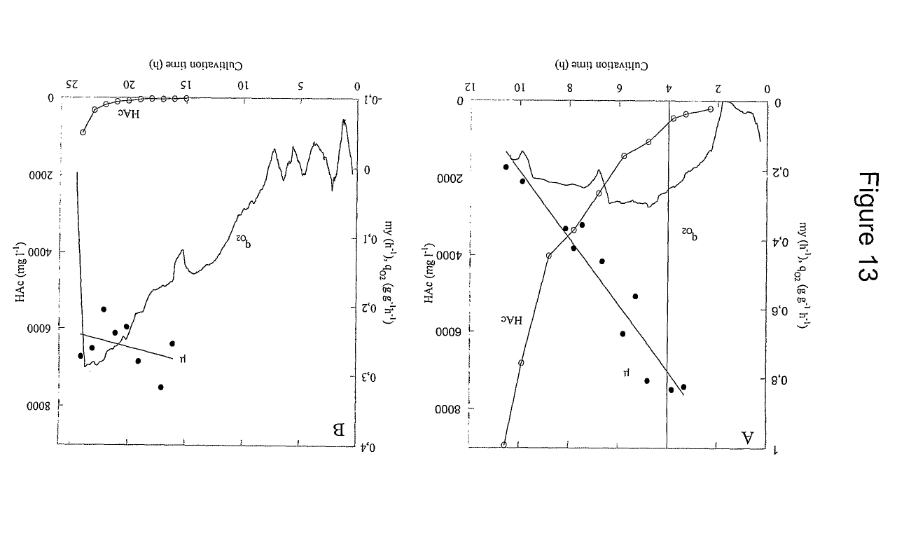

The aim of this example was to find a means to cultivate E. coli cells to high cell density without fed-batch control and without compromising the possibilities for a protein product accumulation. A batch experiment was thus performed where the WT cell accumulation capacity was compared to accumulation of cells from one of the mutants, PTS_{GlcMan,} in batch cultivation. The data are shown in FIG. 13.

The final cell dry weights were 27 and 34 g l⁻¹ for the WT and PTS_{GlcMan}, respectively (data not shown). The WT reaches this value after ten hours while the lower growth rate of the mutant leads to a cultivation time of 25 hours to reach the same cell weight. This is due to the lower glucose uptake rate, which was according to theory.

Under this time the WT cell produced ten times more acetic acid than the mutant i.e. 9000 mg l⁻¹ compared to 900 mg l⁻¹. This high accumulation results in that the growth rate was severely affected very early on in the cultivation. In the beginning the WT culture grew at a growth rate of 0.8 h⁻¹ but at the end the corresponding rate was only 0.2 h⁻¹. It could be seen that already at acetate levels of 500 mg l⁻¹, the growth rate was decreased. At this time the cell concentration was only 3 g l⁻¹. This is in contrast to the cultivation with PTS_{GlcMan} where the specific growth rate was constant over the production interval. The specific oxygen consumption rate reached the highest value very early on in the WT cultivation, as is shown in FIG. 13A. For the mutant strain it took 17 hours longer time to reach the same oxygen consumption rate (FIG. 13B), a figure that is set by the growth rate difference. In FIG. 13A is thus shown, by a vertical line, the principal limit for the batch cultivation of the WT which corresponds to the point in time where the cell is severely affected by the amounts of acetic acid.

The yield coefficient on biomass over glucose added was calculated to 0.39 and 0.34 g g⁻¹ at the end of the cultivation for the WT and PTS_{GlcMan} strains, respectively. This should be compared to the values in the early phase of the cultivation where the yield coefficient was 0.49 and 0.52 g g⁻¹ respectively. The high concentration of the acetate produced by the WT strain accounts for the carbon loss and explains the low yield. The mutant on the other hand, does not produce acetate to the extent of the WT cell but still has a low yield.

Furthermore, as these cells are not producing the recombinant product, this is not the reason for the reduced yield coefficients in the end of the cultivations. It seems likely that an increased maintenance might occur from the cellular conflict of having a combination of a high glucose concentration in the medium and non-functional parts of the PTS system, which might explain the lowered yield coefficients. Even though GalP and the Mgl transport systems are believed to function only at low glucose concentrations (Ferenci T, 1996), the cell might still try to induce this pathway to gain carbon and energy.

The cultivation with the WT was thus eventually limited by acetate accumulation, which severely reduced the growth rate whereas PTS_{GlcMan} was limited by the oxygen transfer. This was clearly evident from the dissolved oxygen data where the WT values were only marginally lowered, DOT=52% but where the corresponding values for the mutant was a DOT=10%.

Discussion

Strains with mutations in the glucose- and mannose specific enzymes II were already in 1975 shown by Curtis and Epstein to have prolonged doubling times (Curtis S and Epstein W, 1975). However, a mutation in II^{Man} did not affect the doubling time compared to the WT, whereas mutations in II^{Glc} or both mutations lead to two to almost five times longer doubling times, respectively (Duetz W et al, 2000). Our data confirm the data of the literature for the WT as well as the mutants lacking enzyme II^{Man} and II^{Glc}. However, the double mutant has three times the doubling time of the WT according to our experiments. This is a considerable higher growth rate than shown by Curtis and Epstein. Since there are no medium differences we draw the conclusion that this is probably a result from unknown differences between the different origins of the strains, AF1000 compared to AB259 [Sandén A M et al, 2002 and Duetz W et al, 2000). Picon et al have since this time reported that the reduced growth rate is a result of a reduced uptake rate of glucose (Picon A et al, 2005).

In this example we have shown that a set of PTS mutants are able to reduce growth, respiration and acetate formation in the same way as a corresponding WT cell cultivated in fed-batch mode however under growth from a simple batch cultivation protocol.

We have shown that in a batch reactor up to 34 g/L of cells could be reached without oxygen supplementation where the DOT at harvest was 10% and where the reactor had a K_{L}a of approximately 800 h⁻¹. The cells are however equally well suited for cultivation on microtitre plates as well as bioreactors. Microtitre plates have been described to reach a K_{L}a of approximately 130-188 h⁻¹ (Duetz W et al, 2000, John G T et al, 2003). Given otherwise equal conditions this implies that the PTS_{GlcMan} strain could reach a cell density of approximately 6.8 g l⁻¹ considering a mean value of the lower oxygen transfer coefficient, and if a DOT of 10% is considered the limit for successful production. If the amount of product enriched was proportional to our example, which was the lowest production rates of all our mutants, a total product amount of 2.2 mg would be the result from cultivation in a 5 ml volume. This is furthermore achieved without the accumulation of acetic acid. This is a result of the fact that the mutants in this paper produce only 10% of the acetic acid of the WT strain under the otherwise same conditions.

A comparison of FIGS. 13A and B gives at hand that already at a comparatively low acetic acid concentration i.e. of 500 mg/L there is a setback in the specific growth rate. At this time the cell concentration is approximately 3 g/L. The use of our strain in this case allows the accumulation of 10 times higher cell mass without a reduced growth rate. In the publication of Jensen and Carlsen (Jensen E and Carlsen S, 1990) it was shown the detrimental effects of acetic acid on the accumulation of recombinant human growth hormone (rhGH). At acetate concentrations of 6 g l⁻¹ growth was inhibited, but already at 2.4 g l⁻¹ the specific production rate of rhGH was decreased. It should be noted that these authors did not test concentrations between 0.1 and 2.4 g,l⁻¹ and that also for this product there might be effects seen at even lower concentrations.

A reduction of acetate formation by mutation of the ptsG gene (enzyme II^{Glc}) was reported earlier (Chou C et al, 1994). Also in this report the mutation lead to increased biomass and recombinant protein (β-galactosidase) concentration by 50%, which for the former corresponds to an increase from approximately 13 to 19 g l⁻¹. Cell engineering has also been used to replace the glucose PTS system by overexpression of GalP or alternatively, overexpression of the regulatory protein Mlc (Kimata K et al, 1998, De Anda R et al, 2006, and Cho S et al, 2005).

The use of the PTS mutants in batch cultivation, lead to accumulation of recombinant product to 13-30% of the total protein and prolonged production periods at maximum production rate. This is a result of the possibility to change the substrate uptake rate. However, except for the productivity the product quality, with respect to the accumulation of full-length active protein, is also very important. The protein production rate from this expression system is coupled to the growth rate, which is seen in FIG. 11. The higher the growth rates the higher the initial production. This was earlier also found for other proteins and other expression systems (Sandén A M et al, 2002, Boström M et al, 2004). However, expression of a recombinant protein at high growth rates was shown to increase the production of a modified protein product (Ryan W et al, 1996). At a growth rate of 0.2 h⁻¹, about 45% of the product was correct and this amount was decreased to 30% when the growth rate increased to 0.5 h⁻¹. We have also been able to show that the degree of proteolysis and inclusion body formation becomes higher the higher the growth rate (Sandén A M et al, 2005) which means that the higher the growth rate the lower the proportion of soluble protein product. It seems thus that a high productivity is inversely proportional to the final protein quality. This makes traditional batch production unsuitable for many products sensitive to these specific conditions. The possibility of reducing the growth rate by use of the mutants described in this paper is thus not only important for accumulation of a high cell density but also for the creation of conditions which might increase the amount of full length and active, soluble product. For a given expression system we will thus need a range of growth (or rather substrate uptake) rates to find an optimal solution for a particular product.

We have chosen β-galactosidase as the model protein in this work since this is a protein, which is naturally present in E. coli, it is generally soluble and we have not experienced any proteolytic degradation despite many years of research and commercial production under a multifold of production conditions. There should thus be no post translational effects on production from e.g. complicated folding pathways. We have also chosen to express the protein from a low copy number vector which all together means that product formation and accumulation should only be affected by the transcription rate from the lacUV5 promoter either in a constitutively expressed mode (lacI_{q}) or by IPTG induced expression since translation is always the same. However, the deletion of enzyme IICB^{Glc} (ptsG) from the glucose specific uptake system in the cytoplasmic membrane leads to a further effect on the expression, which is not the case with the IIAB^{Man} deletion (manX gene). This is exemplified in FIG. 11 where the production rate during constitutive production is shown for the respective mutations. During these experiments on minimal salts medium only very low production rates were achieved when the cells lack the ptsG gene. This effect was restored by addition of yeast extract. A comparison of FIGS. 11 and 12 further shows that if production is induced instead of being constitutively expressed, production is also in this case restored although the high production rate was reduced some time earlier compared to the WT strain.

The lacUV5 promoter is theoretically not subjected to catabolite repression and additions of cAMP in separate experiments (data not shown) did indeed not influence the production rate. Additions of amino acids and attempts to induce stringent control and guanosine tetraphosphate (ppGpp) formation to affect transcription were unsuccessful (data not shown). The lowered production levels of the PTS_{Glc} and PTS_{GlcMan} strains in minimal medium are thus likely an unknown control effect of the chosen promoter with respect to the repression mechanism (lacI_{q}) and the lack of IICB^{Glc}.

It is clear that glucose and carbon uptake is tightly controlled in E. coli and a large part of this control lies on the glucose permease. A deletion of this enzyme might seriously interrupt the balance of control of carbon uptake and possibly also the further metabolism. The phosphorylation status of this enzyme controls catabolite repression but also the control affected by the protein Mlc (Kimata K et al, 1998, Plumbridge J, 1998). This protein further control the expression of the some of the PTS genes such as: ptsHI, ptsG and manXYZ (Kimata K et al, 1998, Plumbridge J, 1998, Plumbridge J, 1999) through depression. At high glucose concentrations IIB^{Glc} is unphosphorylated and this leads to increased concentrations of free Mlc, which is generally bound to the phosphorylated enzyme. When IICB^{Glc} is deleted, Mlc is obviously not bound to the protein and this should theoretically lead to decreased production of the present PTS enzymes, in turn leading to decreased glucose uptake. Our hypothesis was that if IICB^{Glc} expression were restored, Mlc would be able to bind the phosphorylated form of IICB^{Glc} and thus relieve the repression of the PTS-genes. We thus investigated if overproduction of IICB^{Glc} was enough to restore the production of β-galactosidase. The ptsG gene was thus expressed from a plasmid under the control of the arabinose promoter in the PTS_{Glc} strain. However, production could not be gained despite inducer concentrations in the range of 60 μM-50 mM. This further supports the conclusion that the control lies on the transcription of the lacUV5-promoter but by an unknown mechanism.

We have suggested the strains described here to be useful in rapid screening for small amounts of target protein in drug discovery. There are further production events where the fed-batch concept limits the number of production methods tested and that is during the phase where protein production processes are developed for later scale up to industrial scale. In order to optimize the production process, ideally the same conditions should be used both in development and production. This can only be accomplished if the feed addition rate is the same. By using the mutants described in this paper process development could also be taken to a lower volume scale and a larger multitude of techniques could be tested in the same period of time. The best systems with respect to productivity and product quality could be scaled up to production where fed-batch is used in a way, which corresponds to the substrate uptake rate of the mutant.

### REFERENCES

1. Picon A et al (2005). Reducing the Glucose Uptake Rate in Escherichia coli Affects Growth Rate But Not Protein Production. Biotechnology and Bioengeneering, vol 90, no. 2, April 2005

2. Moat A. G et al (2002). Microbial Physiology, Chapter 5.

3. Postma P. W et al (1996). Escherichia coli and Salmonella. Cellular and molecular biology. Phosphoenolpyruvate: Carbohydrate Phosphotransferase System. Chapter 75

4. Enfors S O and Häggström L (2000). Bioprocess Technology, Fundamentals and applications, KTH, Stockholm

5. Reznikoff W. S and Abelson J. N (1980). The Operon, The lac Promoter

6. Sandén A. M (2004). Impact of glucose feed rate on productivity and recombinant protein quality in Escherichia coli. Doctoral Thesis Stockholm, Sweden

7. Lengeler J. W et al (1999). Biology of the procaryotes

8. Boström M. (2004). Design of substrate induced transcription for control of recombinant protein production in Escherichia coli. Doctoral Thesis Stockholm, Sweden

9. Sandén A. M et al (2002). Limiting factors in Escherichia coli fedbatch production of recombinant proteins. Wiley Interscience (online, www.interscience.wiley.com)

10. Gosset G (2005). Improvement of Escherichia coli production strains by modification of the phosphoenolpyruvate:sugar phosphotransferase system. Universidad Nacional Autonoma de Mexico, Apdo

11. Ferenci T (1996). Adaptation to life at micromolar nutrient levels: the regulation of Escherichia coli glucose transport by endoinduction and cAMP. Fems microbiology reviews

12. Kalnins A et al (1983). Sequence of the lacZ gene of Escherichia coli. The Embo Journal Vol. 2 no. 4

13. Sandén A M, Boström M, Markland K, Larsson G (2005) Solubility and proteolysis of the Zb-MalE and Zb-MalE31 proteins during overproduction in Escherichia coli. Biotech Bioeng, 90:239-247.

14. Boström M, Markland K, Sandén A M, Hedhammar M, Hober S, Larsson G (2005) Effect of substrate feed rate on recombinant protein secretion, degradation and inclusion body formation in Escherichia coli. Appl Microbiol Biotechnol, 68:82-90.

15. Linn T, St. Pierre R (1990) Improved vector system for constructing transcriptional fusions that ensures independent translation of lacZ. J Bacteriol, 172:1077-1084.

16. Rose R (1998) The nucleotide sequence of pACYC184. Nucl Acid Res, 16:355.

17. Larsson G, Törnkvist M (1996) Rapid sampling, cell inactivation and evaluation of low extracellular glucose concentrations during fed-batch cultivation. J Biotechnol, 49:69-82.

18. Doelle H, Ewings K, Hollywood N (1982) Regulation of glucose metabolism in bacterial systems. Adv Biochem Eng, 23:1-35.

19. Meyer H-P, Leist C, Fiechter A (1984) Acetate formation in continuous culture of Escherichia coli K12 D 1 on defined and complex media. J Biotechnol, 1:355-358.

20. Curtis S, Epstein W (1975) Phosphorylation of D-glucose in Escherichia coli mutants defective in glucosephosphotransferase, mannosephosphotransferase, and glucokinase. J Bacteriol, 122:1189-1199.

21. Duetz W, Rüedi L, Hermann R, O'Connor K, Büchs J, Witholt B (2000): Methods for intense aeration, growth, storage, and replication of bacterial strains in microtiter plates. Appl Env Micro, 66:2641-2646.

22. John G T, Kliman I, Wittman C, Heinzle E (2003) Integrated optical sensing of dissolved oxygen in microtitre plates: a novel tool for microbial cultivation. Biotech Bioeng, 81:829-836.

23. Jensen E, Carlsen S (1990) Production of recombinant human growth hormone in Escherichia coli: expression of different precursors and physiological effects of glucose, acetate and salts. Biotech Bioeng, 36:1-11.

24. Chou C, Bennett G, San K (1994) Effect of modified glucose uptake using genetic engineering techniques on high-level recombinant protein in Escherichia coli dense cultures. Biotechnol. Bioeng, 44:952-960.

25. Kimata K, Inada T, Tagami H, Aiba H (1998) A global repressor (Mlc) is involved in glucose induction of the ptsG gene encoding major glucose transporter in Escherichia coli. Mol Microbiol, 29:1509-1519.

26. De Anda R, Lara A, Hernandez V, Hernandez-Montalvo V, Gosset G, Bolivar F, Ramirez O (2006) Replacement of the glucose phosphotransferase transport system by galactose permease reduces acetate accumulation and improves process performance of Escherichia coli for recombinant protein production without impairment of growth rate. Metabolic Eng 2006, 8:281-290.

27. Cho S, Shin D, Ji G E, Heu S, Ryu S (2005) High-level recombinant protein production by overexpression of Mcl in Escherichia coli. J Biotechnol, 119:197-203.

28. Boström M, Larsson G (2004) Process design for recombinant protein production based on the promoter, PmalK. Appl Microbiol Biotechnol, 66:200-208.

29. Ryan W, Collier P, Loredo L, Pope J, Sachdev R (1996) Growth kinetics of Escherichia coli and expresiion of a recombinant protein and its isoforms under heat shock conditions. Biotechnol. Prog. 12:596-601.

30. Plumbridge J (1998) Control of the expression of the manXYZ operon in Escherichia coli: Mlc is a negative regulator of the mannose PTS. Mol Microbiol 27:369-380

31. Plumbridge J (1999) Expression of the phosphotransferase system both mediates and is mediated by Mlc regulation in Escherichia coli. Mol Microbiol 33:260-273

1. Method for multiparallel construction of host/vector-systems, comprising the transformation of a plurality of host strains, wherein substantially all host strains have deleterious mutations in at least one uptake system for a critical substrate needed for the growth of said host strain, with a plurality of vectors encoding at least one protein, and expressing said recombinant protein in said host strains.

2. Method according to claim 1, wherein the host strains are a prokaryotic organism.

3. Method according to claim 1, wherein the host strains are E. coli.

4. Method according to claim 3, wherein at least one host strain has a deleterious mutation in ptsG and at least one host strain has a deleterious mutation in ptsM.

5. Method according to claim 4, wherein at least one host strain has at least one deleterious mutation in both ptsG and ptsM.

6. Method according to claim 1, wherein in the plurality of vectors individually comprise an origin of replication, a promoter and a protein encoding sequence and optionally a signal peptide, a reporter protein, a purification tag and/or an antibiotic resistance gene.

7. Method according to claim 1, wherein at least one vector comprises an inducible promoter directing the expression of a member of an uptake system for a critical substrate, wherein said member is deleteriously mutated in said host strain.

8. Kit for performing the method according to claim 1, comprising said plurality of host strains, wherein substantially all host strains have deleterious mutations in at least one uptake system for a critical substrate needed for the growth of said host strain, and optionally a plurality of vectors comprising an origin of replication, a promoter and a protein encoding sequence or a site for inserting a protein encoding sequence and optionally a signal peptide, a reporter protein, a purification tag and/or an antibiotic resistance gene.

9. Host/vector-system obtainable by the method according to claim 1.

10. (canceled)

11. A method of producing a recombinant protein, comprising expressing a recombinant protein in a host/vector-system according to claim 9.

12. Kit according to claim 8, wherein the host strains are a prokaryotic organism.

13. Kit according to claim 8, wherein the host strains are E. coli.

14. Kit according to claim 13, wherein at least one host strain has a deleterious mutation in ptsG and at least one host strain has a deleterious mutation in ptsM.

15. Kit according to claim 14, wherein at least one host strain has at least one deleterious mutation in both ptsG and ptsM.

16. Kit according to claim 8, wherein the plurality of vectors individually comprise an origin of replication, a promoter and a protein encoding sequence and optionally a signal peptide, a reporter protein, a purification tag and/ or an antibiotic resistance gene.

17. Kit according to claim 8, wherein at least one vector comprises an inducible promoter directing the expression of a member of an uptake system for a critical substrate, wherein said member is deleteriously mutated in said host strain.

18. Host/vector-system obtainable by the method according to claim 2.

19. Host/vector-system obtainable by the method according to claim 3.

20. Host/vector-system obtainable by the method according to claim 4.

21. Host/vector-system obtainable by the method according to claim 5.
